(19) United States
(12) Patent Application Publication
(10) Pub. No.: US 2022/0177967 A1
(43) Pub. Date: Jun. 9, 2022

(54) REAGENTS, METHODS AND KITS FOR IDENTIFYING PREGNANT HUMAN BEINGS AT RISK FOR PLACENTAL BED DISORDER(S)

(71) Applicant: EDWARD E. WINGER, M.D., PROFESSIONAL CORPORATION, San Francisco, CA (US)

(72) Inventors: Edward WINGER, San Francisco, CA (US); Jane REED, San Francisco, CA (US)

(73) Assignee: EDWARD E. WINGER, M.D., PROFESSIONAL CORPORATION, San Francisco, CA (US)

(21) Appl. No.: 17/437,100

(22) PCT Filed: Mar. 6, 2020
(86) PCT No.: PCT/US2020/021419

§ 371 Date: Sep. 8, 2021

Related U.S. Application Data
(60) Provisional application No. 62/815,787, filed on Mar. 8, 2019.

(51) Int. Cl. C12Q 1/6883

(52) U.S. Cl. CPC C12Q 1/6883; C12Q 2600/118; C12Q 2600/178; C12Q 2600/158

(57) ABSTRACT

This disclosure provides microRNA (miRNA)-based tests and treatment protocols for identifying and/or treating pregnant human beings at risk for a placental bed disorder during pregnancy, as well as reagents and/or kits relating to the same.

### RELATED APPLICATIONS

This application claims priority to U.S. Ser. No. 62/815,787 filed on Mar. 8, 2019, the entire contents of which are hereby incorporated into this application.

### FIELD OF THE DISCLOSURE

This disclosure provides microRNA (miRNA)-based tests and treatment protocols for identifying and/or treating pregnant human beings at risk for a placental bed disorder during pregnancy, as well as reagents and/or kits relating to the same.

### BACKGROUND OF THE DISCLOSURE

“Preeclampsia-related conditions” represent a group of conditions that together have been considered conditions with a common etiology that include, but are not limited to, pregnancy conditions such as preeclampsia, preterm birth, HELLP Syndrome (a complication of pregnancy characterized by hemolysis, elevated liver enzymes, and a low platelet count), gestational diabetes, miscarriage, implantation failure, fetal growth restriction and premature rupture of the membranes. These conditions arise because of disordered or inadequate transformation of spiral arteries within the endometrium at the site of implantation. Thus, these conditions have been designated placental bed disorders. (Pijnenborg, et al. Placental bed disorders: basic science and its translation to obstetrics. Cambridge University Press, Jun. 3, 2010, ISBN-13: 978-0521517850; ISBN-10: 0521517850).

Preeclampsia, as an example of a placental bed disorder, affects at least 2-3% of all pregnancies and is a major cause of maternal and perinatal morbidity and mortality (Knight, et al. eds. on behalf of MBRRACEUK. Saving lives, improving mothers' care—lessons learned to inform future maternity care from the UK and Ireland confidential enquiries into maternal deaths and morbidity 2009-12. Oxford: National Perinatal Epidemiology Unit, University of Oxford; 2014). The condition is recognized clinically after 20 weeks of gestation with the new appearance of hypertension and proteinuria. In countries with limited access to medical care, it is estimated that the disorder is responsible annually for greater than 60,000 deaths worldwide (World Health Org. 2005. World health report: Make every mother and child count. Geneva: World Health Org. URL: http://www.who.int/whr/2005/whr2005 en.pdf. Last accessed Jul. 24, 2017). In developed countries, therapeutic intervention is often concluded with early delivery. While this intervention protects the mother, it results in significant morbidity and mortality to the neonate Friedman et al. Neonatal outcome after preterm delivery for preeclampsia. Am J Obstet Gynecol. 1995; 172:1785-1792). Early diagnosis has been a goal permitting intervention at an early time point (Bujold, et al. Prevention of preeclampsia and intrauterine growth restriction with aspirin started in early pregnancy: a meta-analysis. Obstet Gynecol. 2010. August; 116(2 Pt 1):402-414).

MicroRNA (miRNA) is a class of RNA species comprising a 22-24 base non-coding polynucleotide. They integrate disparate genetic elements into collaborative metabolic and signaling pathways. They form networks that supervise coordinated expression of mRNAs that guide and maintain cell identity and buffer cell systems against changing conditions. MicroRNA has attracted great interest in the diagnosis and monitoring of various conditions including cancer, autoimmune, inflammatory and neurologic diseases (DePlanell-Saguor, et al. Analytical aspects of microRNA in Diagnostics: a review Analytica Chimica Acta. 2011; 699(2): 134-152). In previous studies, it was determined that first trimester peripheral blood mononuclear cell (PBMC) microRNA provides sensitive and specific prediction of preeclampsia and preterm birth when sampled within a range of 4-14 weeks gestation (Winger et al. Early first trimester peripheral blood cell microRNA predicts risk of preterm delivery in pregnant women: Proof of concept. PLoS One. 2017 Jul. 10; 12(7):e0180124; Winger et al. Peripheral blood cell microRNA quantification during the first trimester predicts preeclampsia: Proof of concept. PLoS One. 2018 Jan. 2; 13(1):e0190654).

While certain miRNA-based tests and treatment protocols for preeclampsia have been developed, there is a need in the art for additional (e.g., more accurate and/or condition-relevant) miRNA-based tests and treatment protocols for placental bed disorders, including preeclampsia. Such miRNA-based tests and treatment protocols are provided by this disclosure.

### BRIEF DESCRIPTION OF THE DRAWINGS

FIG. 1. miRNA signal levels for hsa-miR-4667-3p.

FIG. 2. miRNA signal levels for hsa-miR-1267.

FIG. 3. miRNA signal levels for hsa-miR-7974.

FIG. 4. miRNA signal levels for hsa-miR-563.

FIG. 5. miRNA signal levels for hsa-miR-3190-5p.

FIG. 6. miRNA signal levels for hsa-miR-6792-3p.

FIG. 7. miRNA signal levels for hsa-miR-98-3p.

FIG. 8. miRNA signal levels for hsa-miR-2116-3p.

FIG. 9. miRNA signal levels for hsa-miR-4310.

FIG. 10. miRNA signal levels for hsa-miR-6737-3p.

FIG. 11. miRNA signal levels for hsa-miR-452-5p.

FIG. 12. miRNA signal levels for hsa-miR-5708.

FIG. 13. miRNA signal levels for hsa-miR-580-3p.

FIG. 14. miRNA signal levels for hsa-miR-1238-3p.

FIG. 15. miRNA signal levels for hsa-miR-6782-3p.

FIG. 16. miRNA signal levels for hsa-miR-6889-3p.

FIG. 17. miRNA signal levels for hsa-miR-4666b.

FIG. 18. miRNA signal levels for hsa-miR-455-5p.

FIG. 19. miRNA signal levels for hsa-miR-4485-5p.

FIG. 20. miRNA signal levels for hsa-miR-149-5p.

FIG. 21. miRNA signal levels for hsa-miR-18b-3p.

FIG. 22. miRNA signal levels for hsa-miR-1537-3p.

FIG. 23. miRNA signal levels for hsa-miR-1539.

FIG. 24. miRNA signal levels for hsa-miR-23c.

FIG. 25. miRNA signal levels for hsa-miR-3611.

FIG. 26. miRNA signal levels for hsa-miR-19a-5p.

FIG. 27. miRNA signal levels for hsa-miR-6819-3p.

FIG. 28. miRNA signal levels for hsa-miR-1237-3p.

FIG. 29. miRNA signal levels for hsa-miR-153-3p.

FIG. 30. miRNA signal levels for hsa-miR-6730-3p.

FIG. 31. miRNA signal levels for hsa-miR-6799-3p.

FIG. 32. miRNA signal levels for hsa-miR-190a-5p.

FIG. 33. miRNA signal levels for hsa-miR-144-3p.

FIG. 34. miRNA signal levels for hsa-miR-548a-5p.

FIG. 35. miRNA signal levels for hsa-miR-548ai.

FIG. 36. miRNA signal levels for hsa-miR-1973.

FIG. 37. miRNA signal levels for hsa-miR-6890-3p.

FIG. 38. miRNA signal levels for hsa-miR-6752-3p.

FIG. 39. miRNA signal levels for hsa-miR-4312.

FIG. 40. miRNA signal levels for hsa-miR-6757-3p.

FIG. 41. miRNA signal levels for hsa-miR-32-5p.

FIG. 42. miRNA signal levels for hsa-miR-186-3p.

FIG. 43. miRNA signal levels for hsa-miR-1236-3p.

FIG. 44. miRNA signal levels for hsa-miR-4731-3p.

FIG. 45. miRNA signal levels for hsa-miR-33b-5p.

FIG. 46. miRNA signal levels for hsa-miR-6812-3p.

FIG. 47. miRNA signal levels for hsa-miR-4536-3p.

FIG. 48. miRNA signal levels for hsa-miR-301a-3p.

FIG. 49. miRNA signal levels for hsa-miR-6763-3p.

FIG. 50. miRNA signal levels for hsa-miR-624-3p.

FIG. 51. miRNA signal levels for hsa-miR-590-5p.

FIG. 52. miRNA signal levels for hsa-miR-191-3p.

FIG. 53. miRNA signal levels for hsa-miR-24-1-5p.

FIG. 54. miRNA signal levels for hsa-miR-144-5p.

FIG. 55. miRNA signal levels for hsa-miR-6870-3p.

FIG. 56. miRNA signal levels for hsa-miR-33a-5p.

FIG. 57. miRNA signal levels for hsa-miR-545-3p.

FIG. 58. miRNA signal levels for hsa-miR-19a-3p.

FIG. 59. miRNA signal levels for hsa-miR-6515-3p.

FIG. 60. miRNA signal levels for hsa-miR-551b-3p.

FIG. 61. miRNA signal levels for hsa-miR-3679-3p.

FIG. 62. miRNA signal levels for hsa-miR-141-3p.

FIG. 63. miRNA signal levels for hsa-miR-557.

FIG. 64. miRNA signal levels for hsa-miR-6766-3p.

FIG. 65. miRNA signal levels for hsa-miR-101-3p.

FIG. 66. miRNA signal levels for hsa-miR-1307-5p.

FIG. 67. miRNA signal levels for hsa-miR-219a-5p.

FIG. 68. miRNA signal levels for hsa-miR-340-5p.

FIG. 69. miRNA signal levels for hsa-miR-628-5p.

FIG. 70. miRNA signal levels for hsa-miR-511-3p.

FIG. 71. miRNA signal levels for hsa-miR-192-5p.

FIG. 72. miRNA signal levels for hsa-miR-362-3p.

FIG. 73. miRNA signal levels for hsa-miR-4433a-5p.

FIG. 74. miRNA signal levels for hsa-miR-4500.

FIG. 75. miRNA signal levels for 6820-5p.

FIG. 76. miRNA signal levels for hsa-miR-493-3p.

FIG. 77. miRNA signal levels for hsa-miR-1537-3p.

FIG. 78. miRNA signal levels for hsa-miR-193a-3p.

FIG. 79. miRNA signal levels for hsa-miR-6795-3p.

FIG. 80. miRNA signal levels for hsa-miR-18b-5p.

FIG. 81. miRNA signal levels for hsa-miR-224-5p.

FIG. 82. miRNA signal levels for hsa-miR-132-3p.

FIG. 83. miRNA signal levels for hsa-miR-570-3p.

FIG. 84. miRNA signal levels for hsa-miR-6511b-3p.

FIG. 85. miRNA signal levels for hsa-miR-6818-5p.

FIG. 86. miRNA signal levels for hsa-miR-7-5p.

FIG. 87. miRNA signal levels for hsa-miR-4536-3p.

FIG. 88. miRNA signal levels for hsa-miR-129-1-3p.

FIG. 89. miRNA signal levels for hsa-miR-215-5p.

FIG. 90. miRNA signal levels for hsa-miR-3938.

FIG. 91. miRNA signal levels for hsa-miR-6855-3p.

FIG. 92. miRNA signal levels for hsa-miR-224-3p.

FIG. 93. miRNA signal levels for hsa-miR-4737.

FIG. 94. miRNA signal levels for hsa-miR-582-3p.

FIG. 95. miRNA signal levels for hsa-miR-30d-3p.

FIG. 96. miRNA signal levels for hsa-miR-6796-3p.

FIG. 97. miRNA signal levels for hsa-miR-429.

FIG. 98. miRNA signal levels for hsa-miR-542-3p.

FIG. 99. miRNA signal levels for hsa-miR-185-5p.

FIG. 100. miRNA signal levels for hsa-miR-296-5p.

### SUMMARY OF THE DISCLOSURE

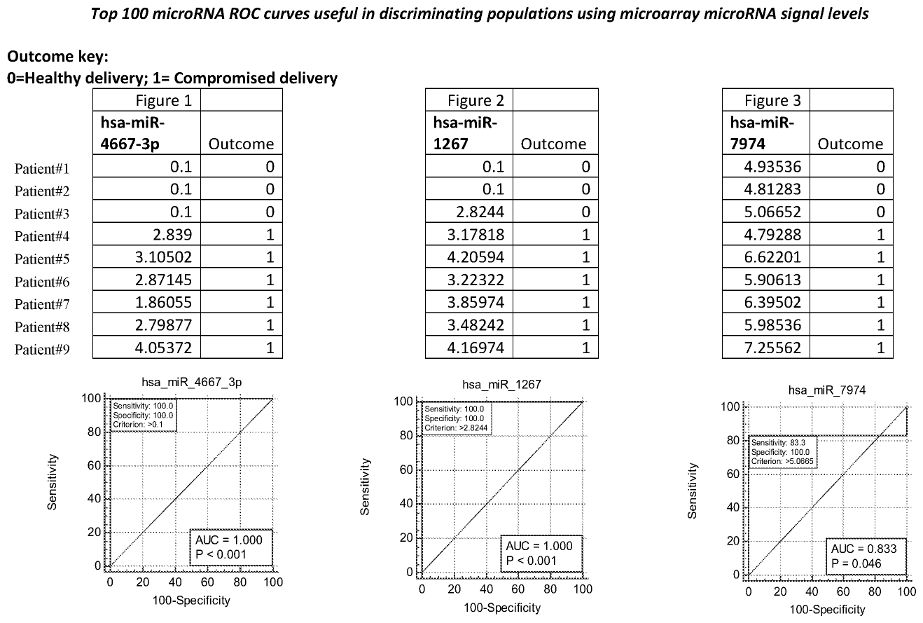

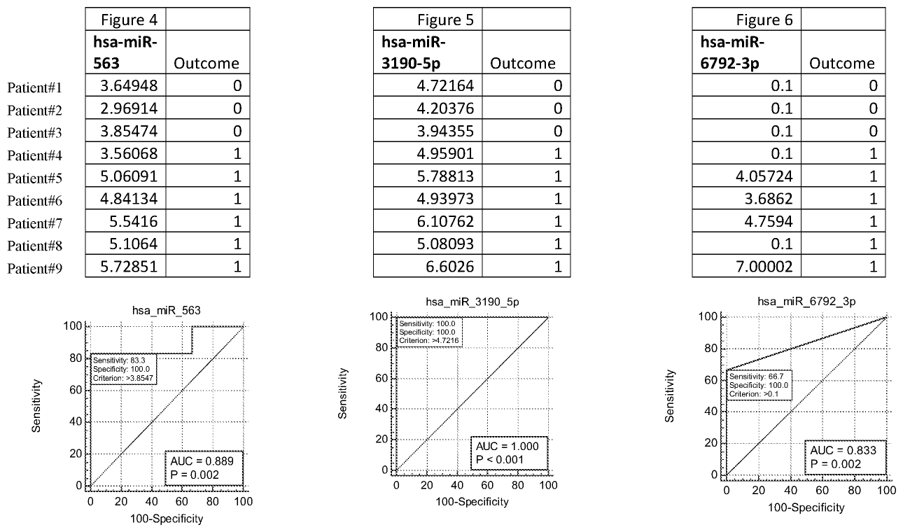

This disclosure provides microRNA (miRNA)-based tests and treatment protocols for identifying and/or treating pregnant human beings at risk for a placental bed disorder during pregnancy, as well as reagents and/or kits relating to the same. In some embodiments, this disclosure provides reagents and methods for identifying at least two characteristic groups in a patient population on the basis of microRNA (miRNA) expression, wherein one characteristic group is associated with a reproductive disorder or a risk of developing such a disorder, comprising the steps of: a) quantifying at least one microRNA from a biological sample derived from immune cells; and, b) segregating the patient population into the groups on the basis of expression of the at least one miRNA, wherein: the miRNA is selected from the group consisting of at least one the miRNAs listed in Table 3, Table 4, Table 5, SEQ ID NOS. 1-100, and/or any of FIGS. 1-100; and/or, the at least one miRNA is selected from the group consisting of hsa-miR-4485-5p, hsa-miR-551b-3p, hsa-miR-24-1-5p, hsa-miR-6819-3p, hsa-miR-1238-3p, hsa-miR-6737-3p, hsa-miR-1237-3p, hsa-miR-6757-3p, hsa-miR-6889-3p, hsa-miR-6752-3p, hsa-miR-191-3p, hsa-miR-6795-3p, hsa-miR-149-5p, hsa-miR-2116-3p, hsa-miR-7974, hsa-miR-23c, hsa-miR-4310, hsa-miR-98-3p, hsa-miR-3190-5p, and/or hsa-miR-4312. In some embodiments, the step of segregating the patient population comprises assigning patients expressing a relatively high level of the at least one miRNA to a first group and assigning patients expressing a relatively low level of the at least one miRNA to a second group. In some such embodiments, the patient population is pregnant human beings and the population segregated in step b) is at risk of developing a placental bed disorder; in some embodiments, the HC ratio is used.

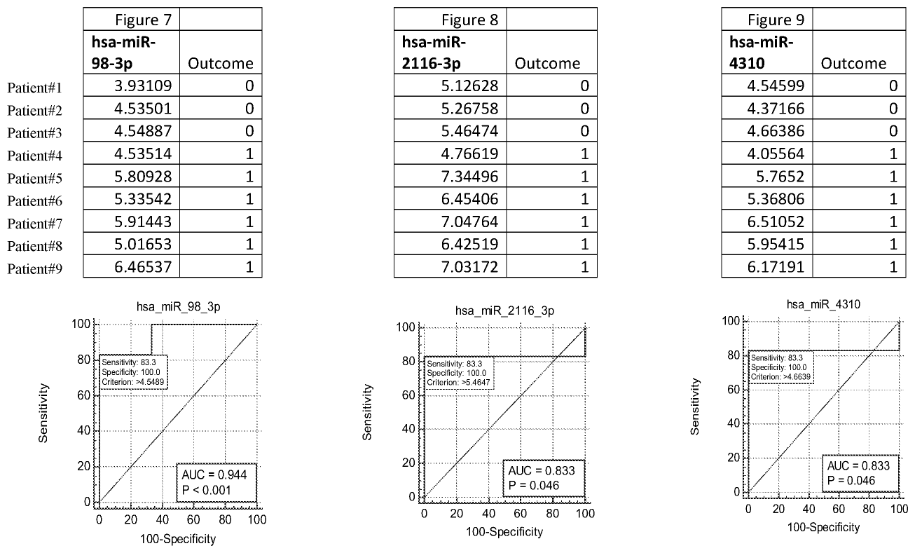

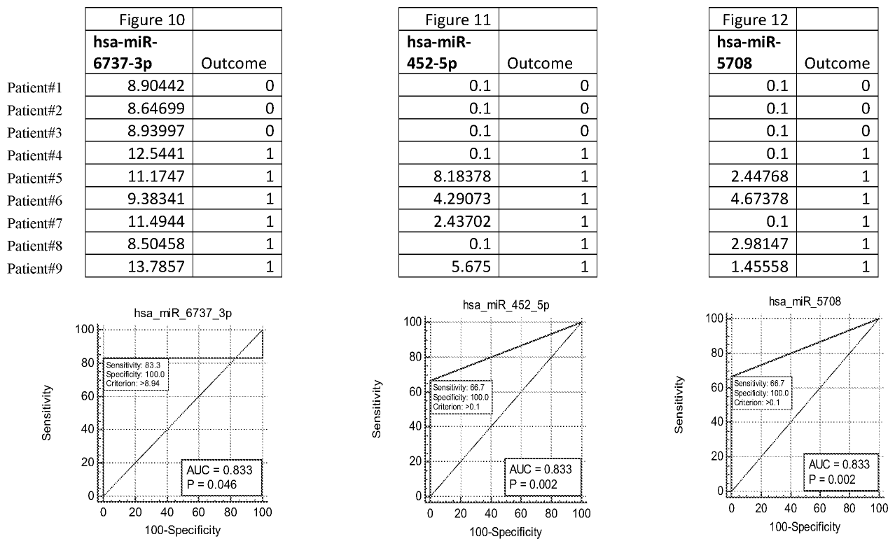

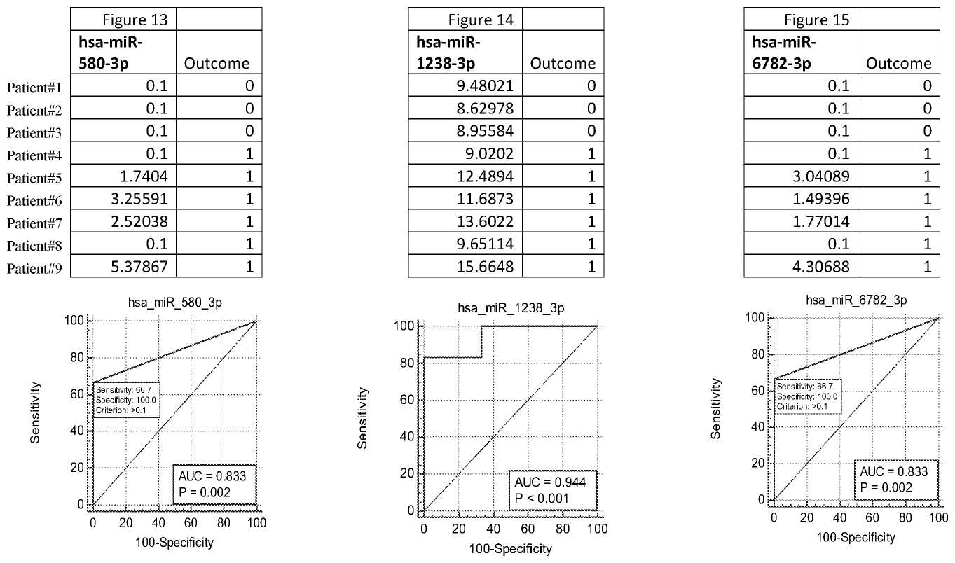

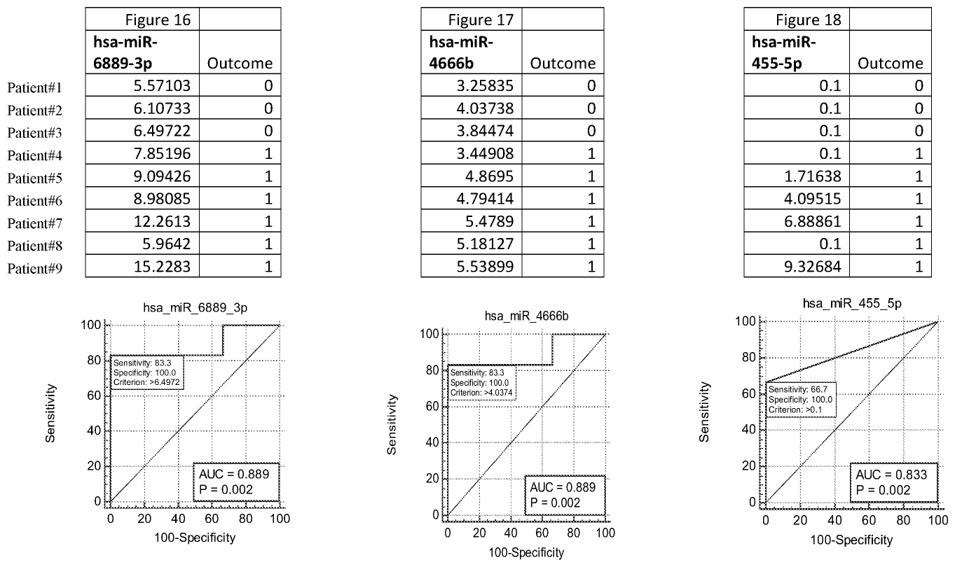

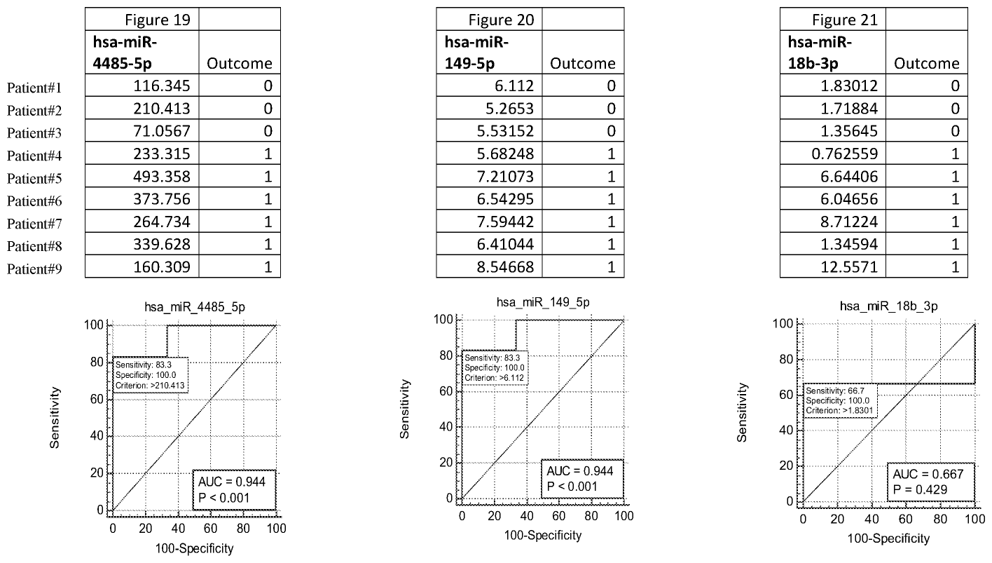

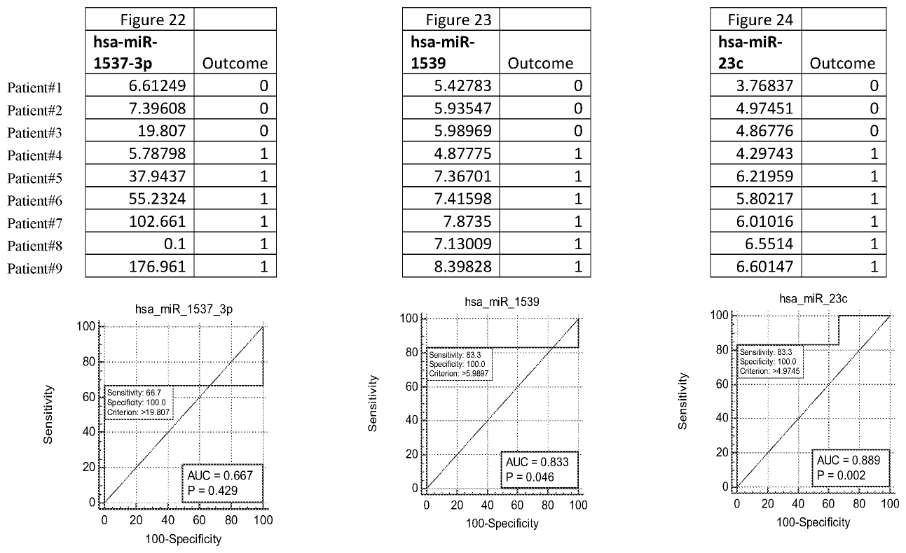

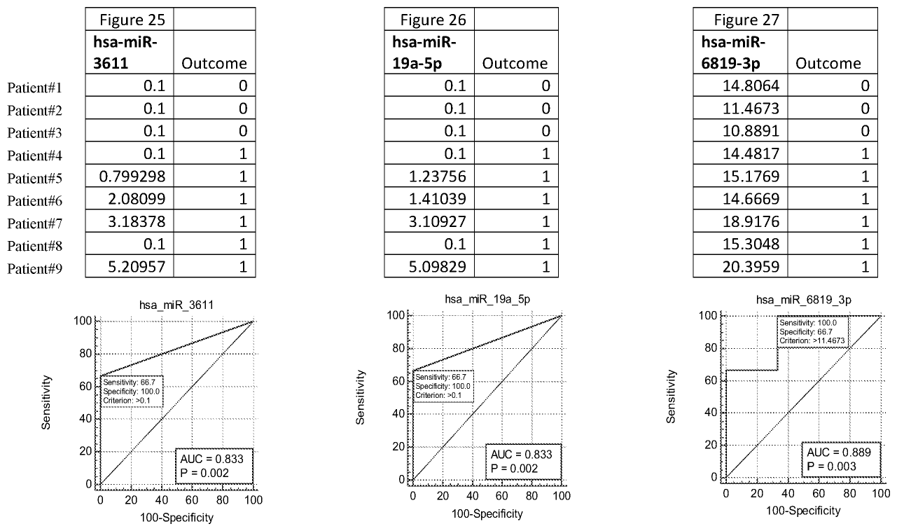

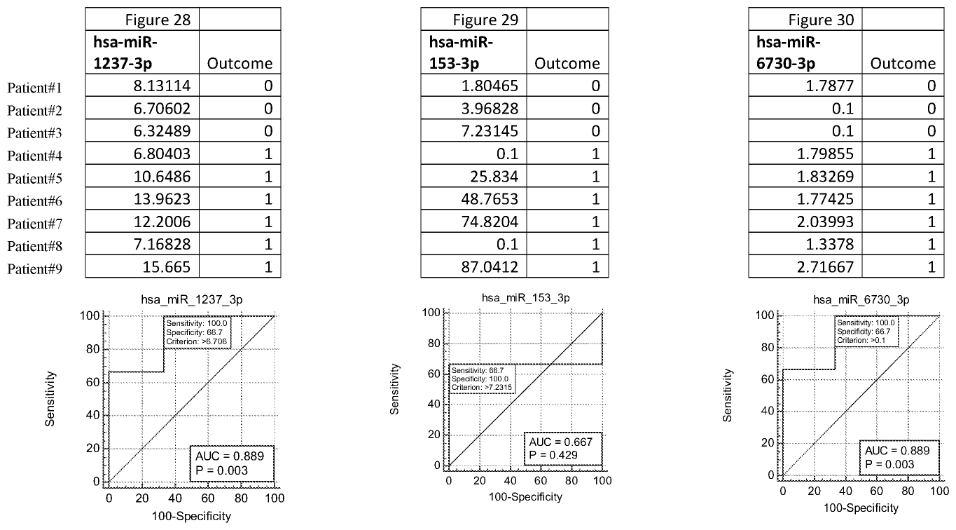

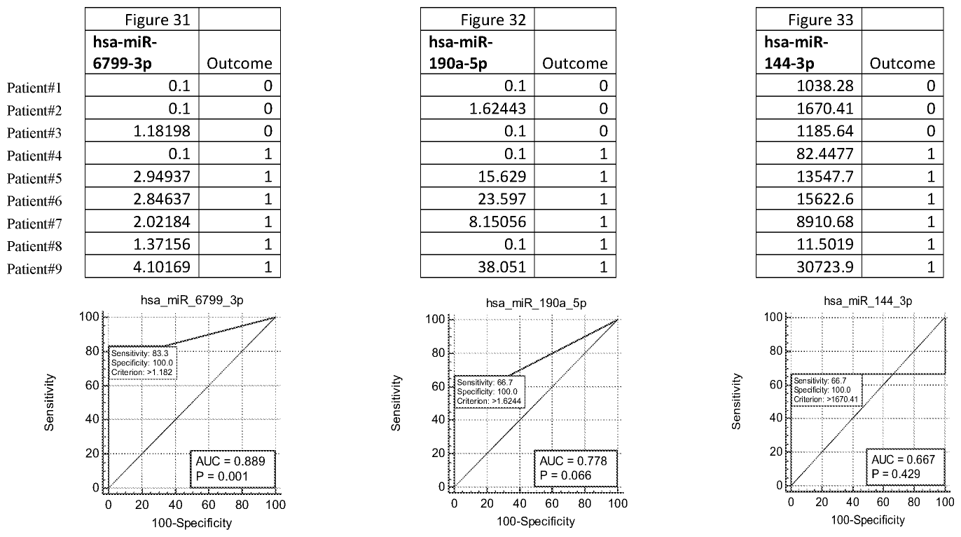

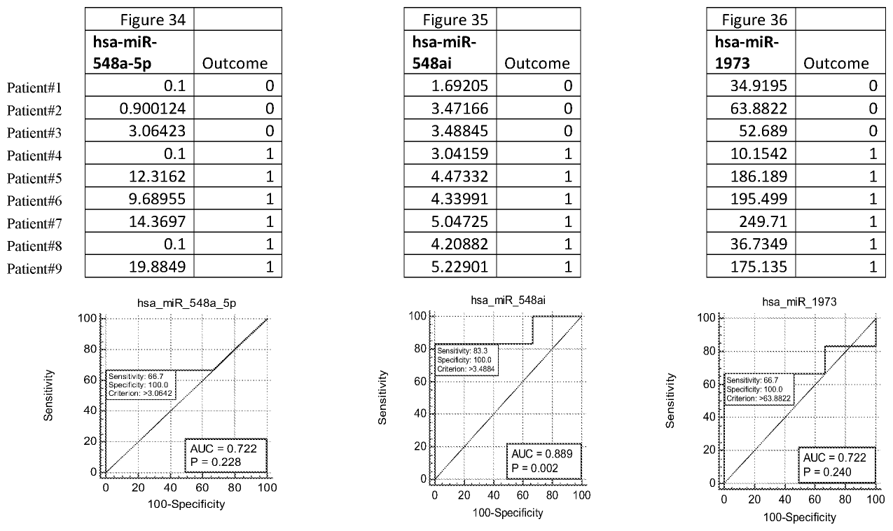

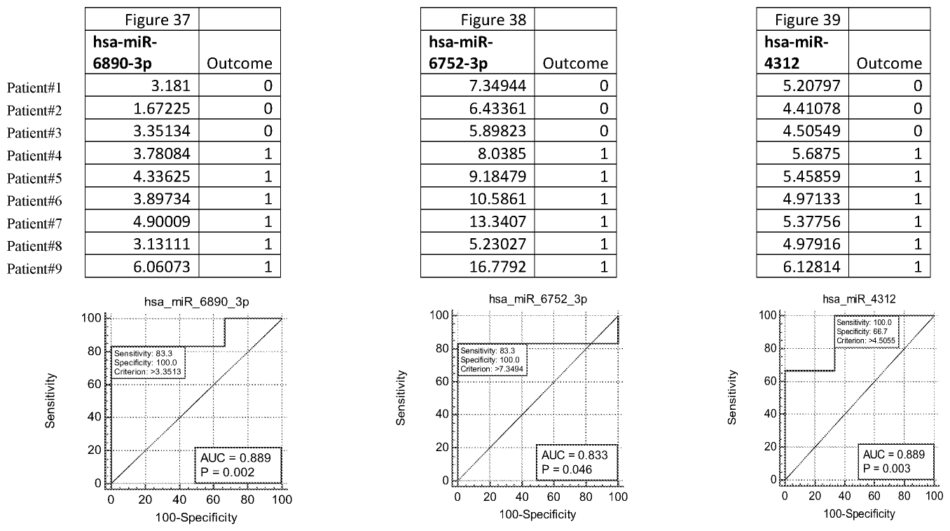

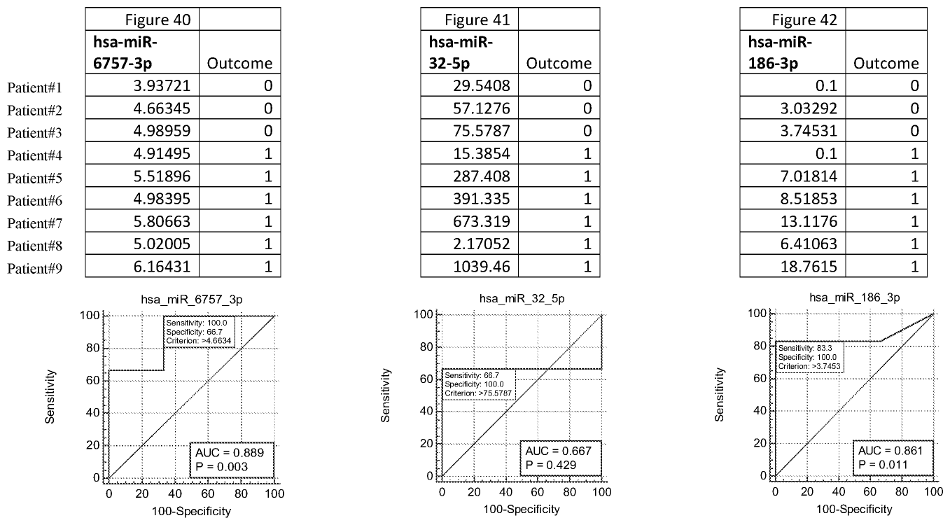

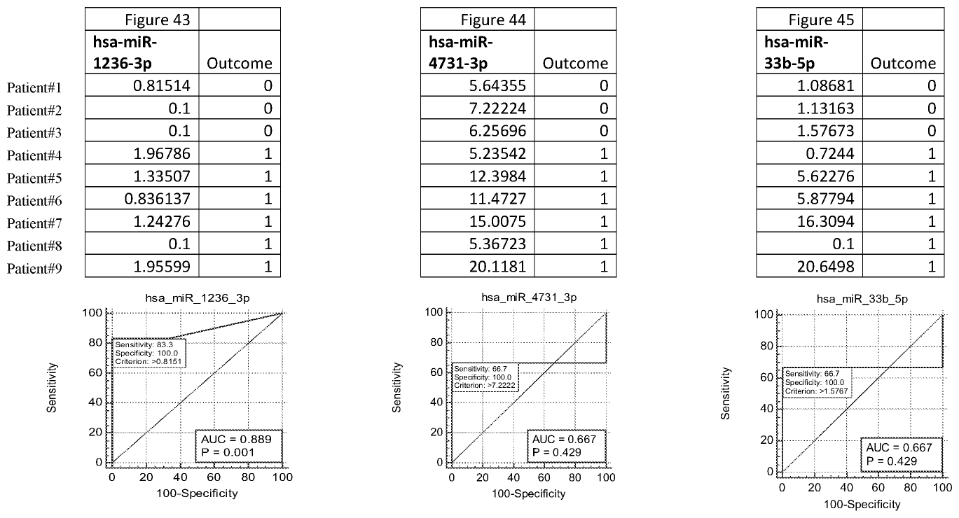

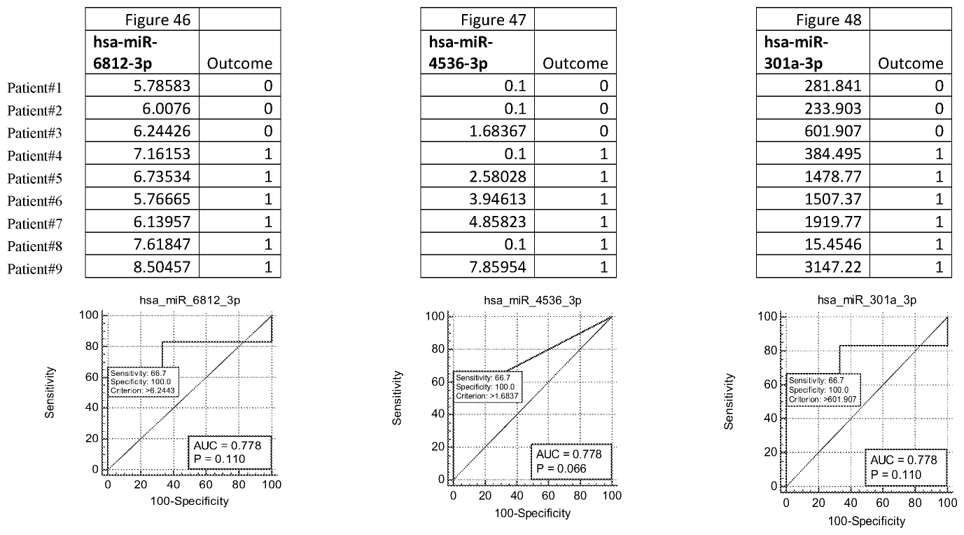

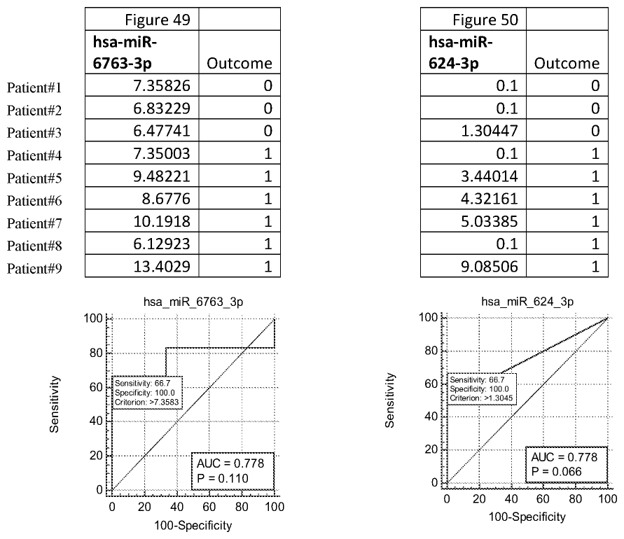

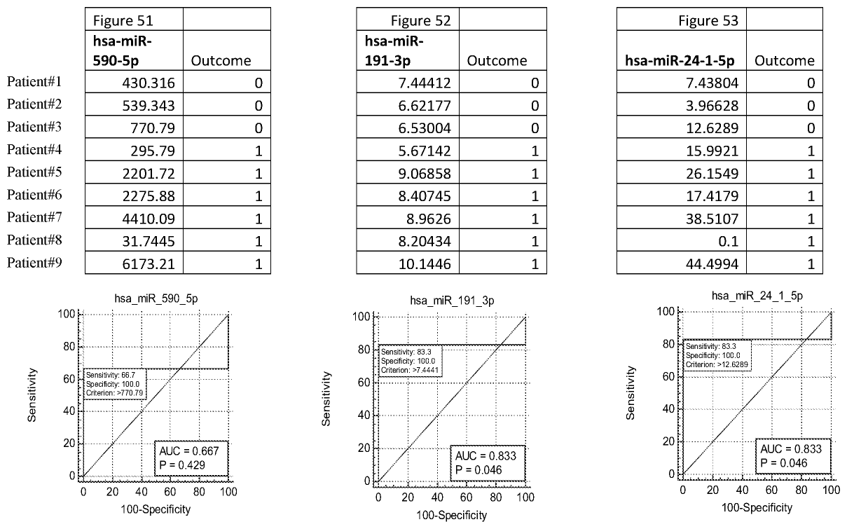

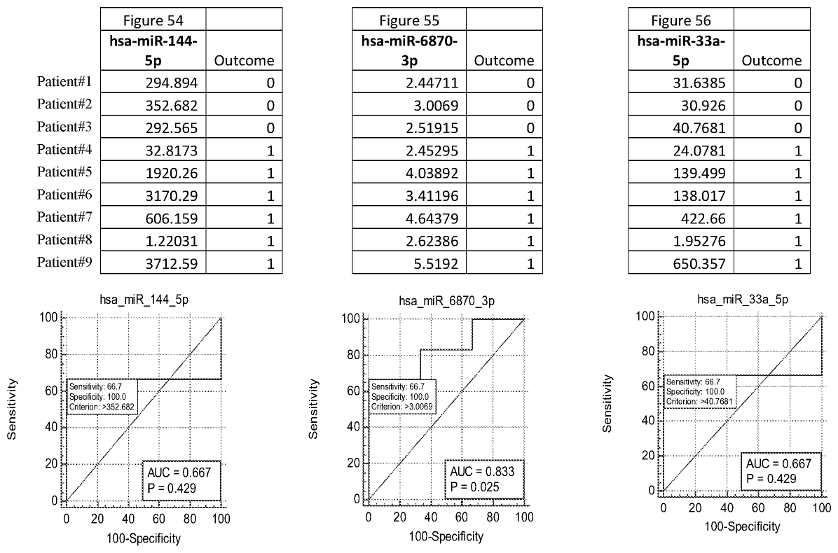

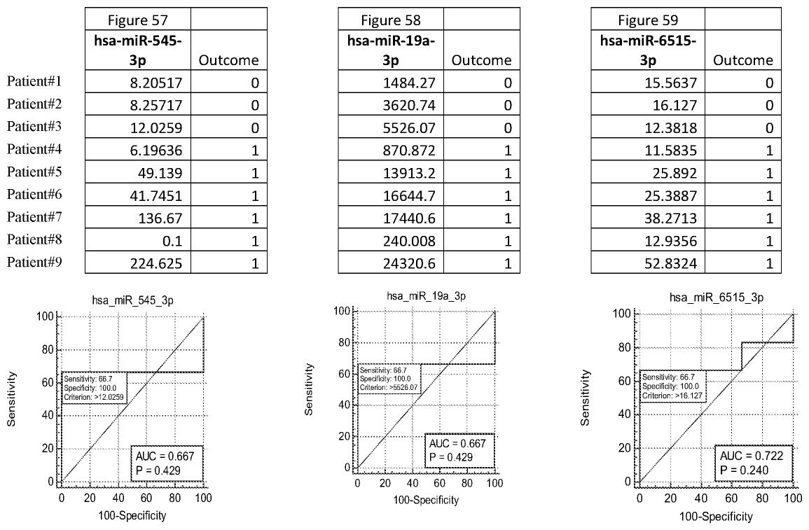

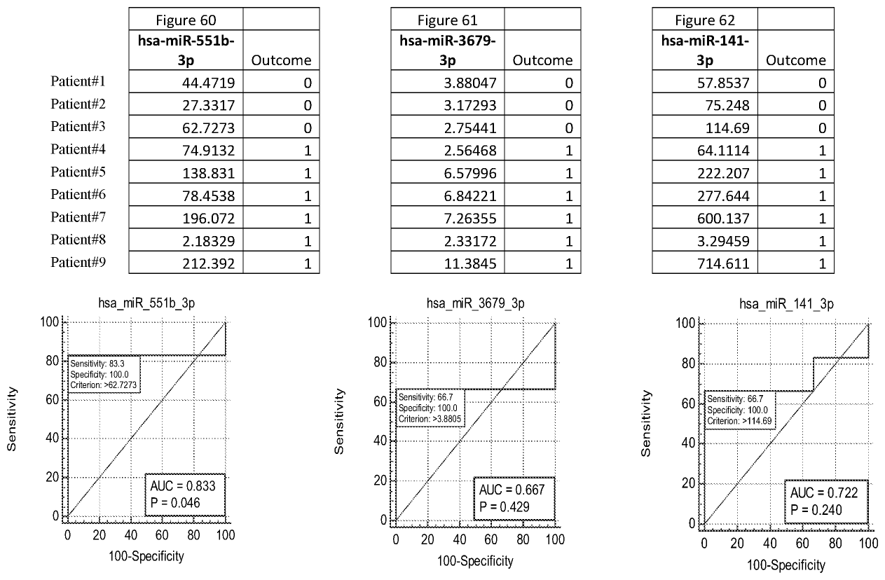

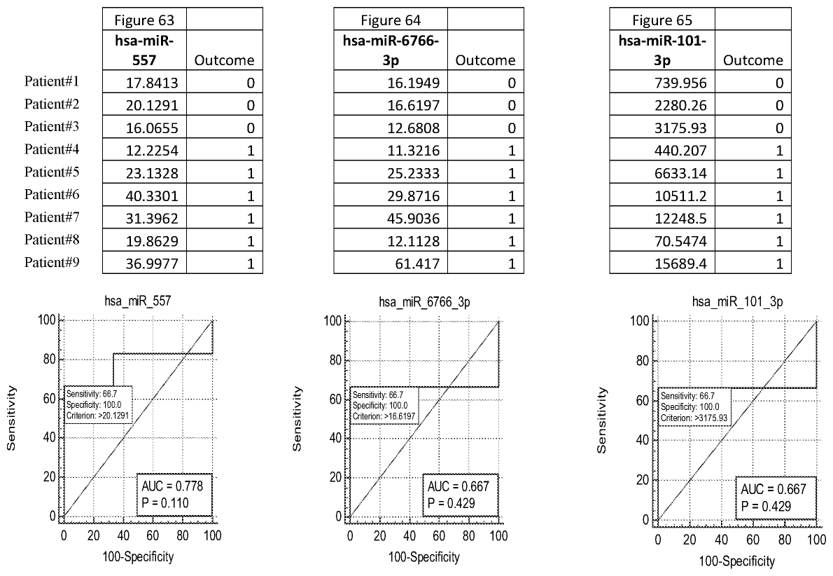

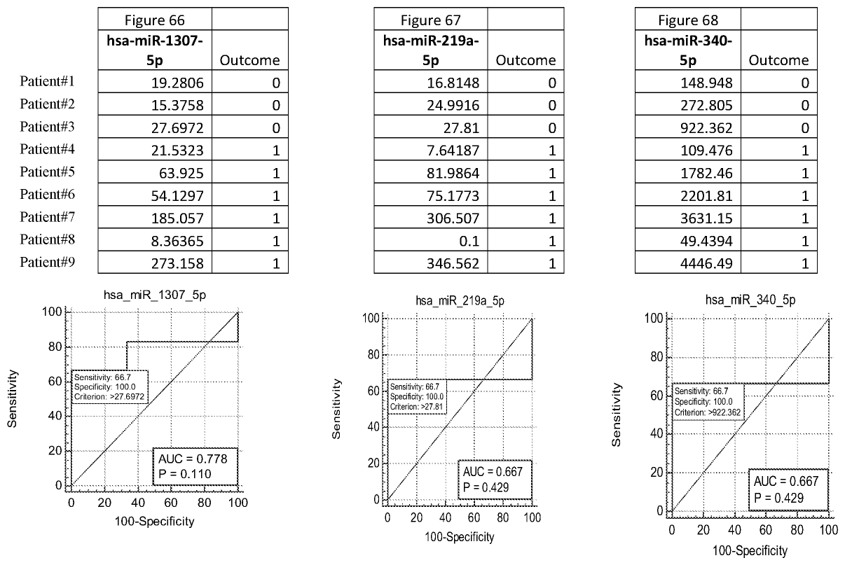

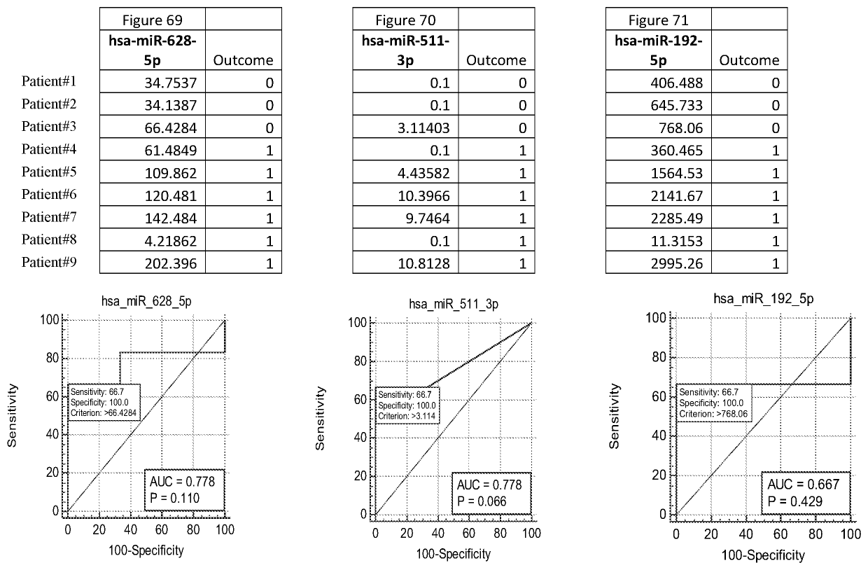

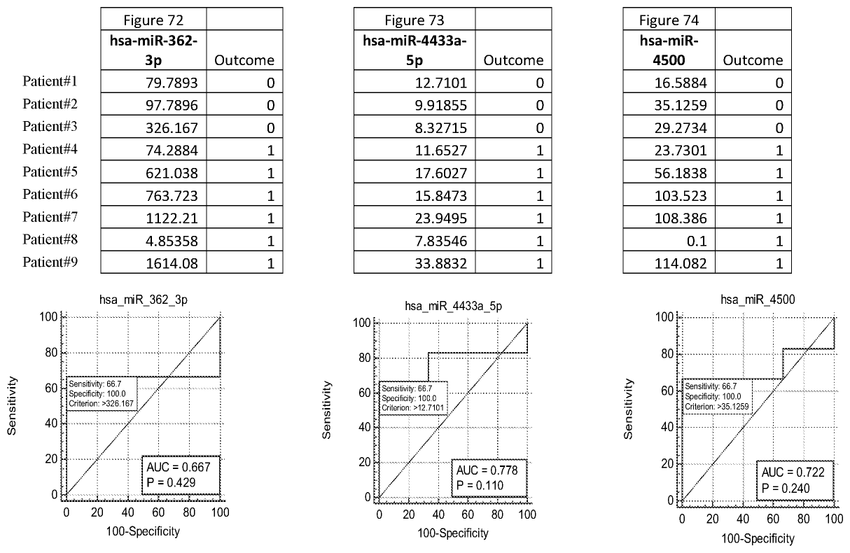

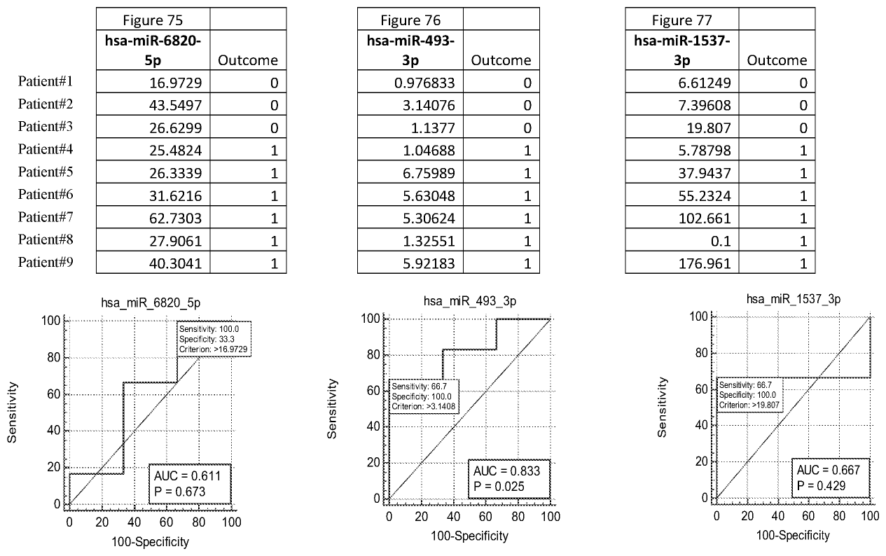

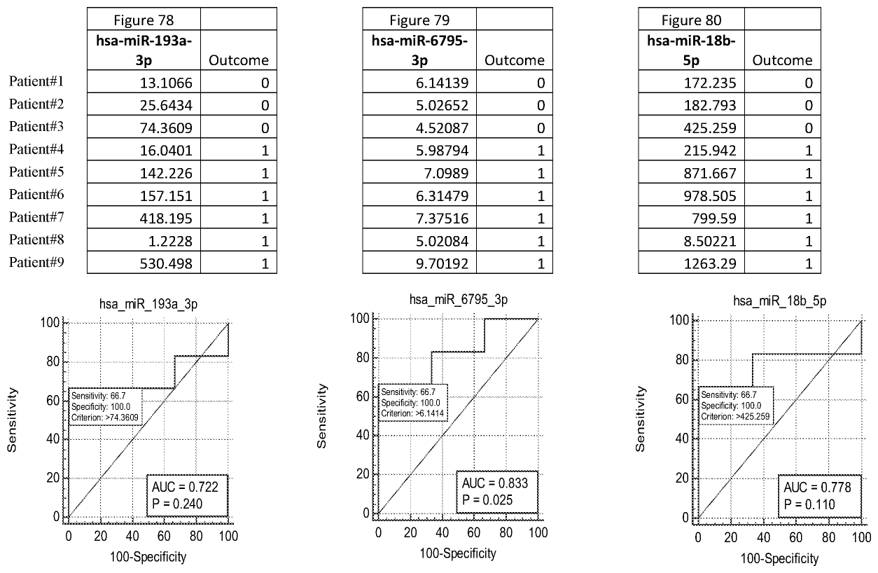

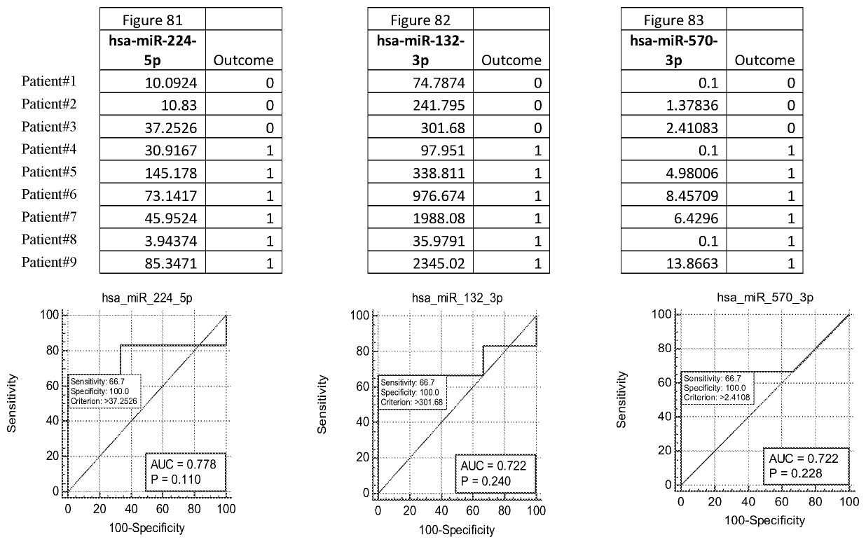

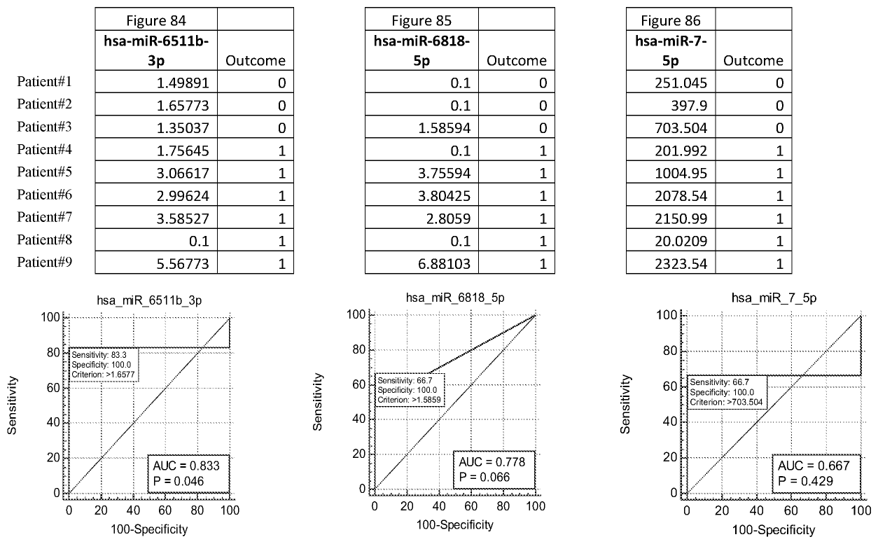

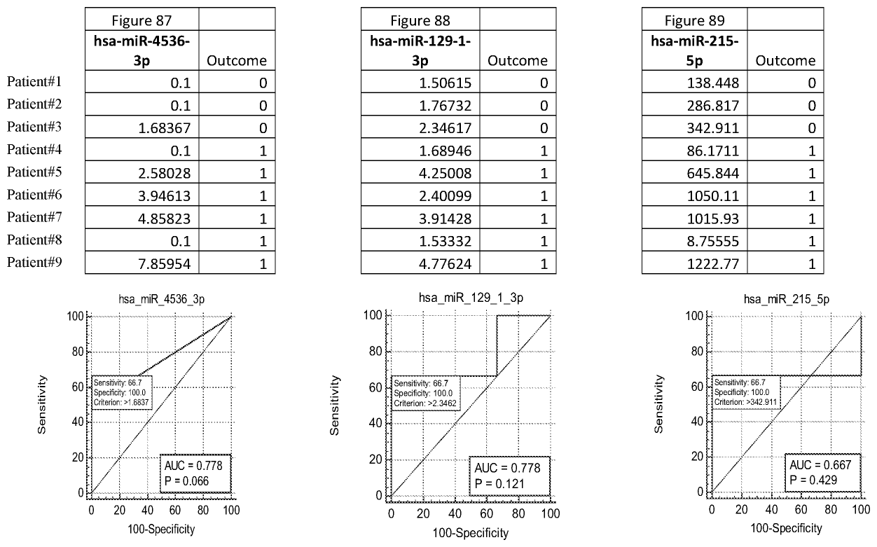

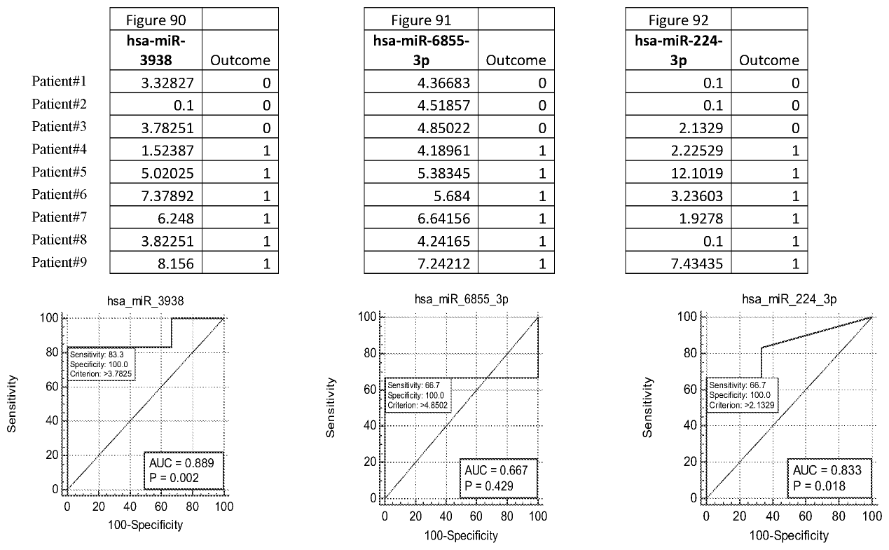

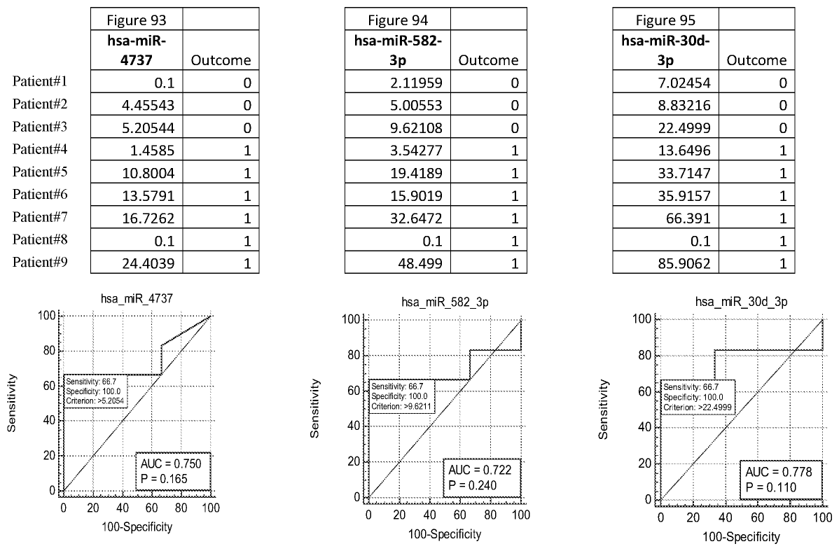

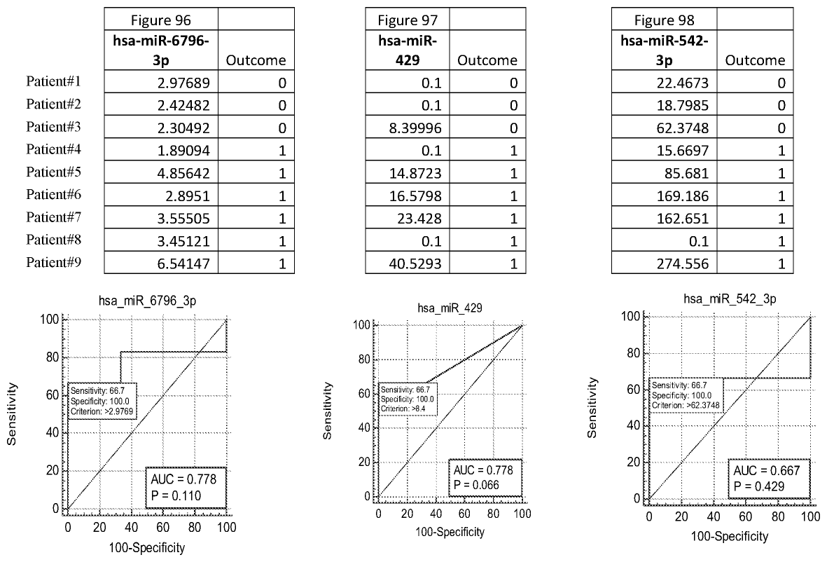

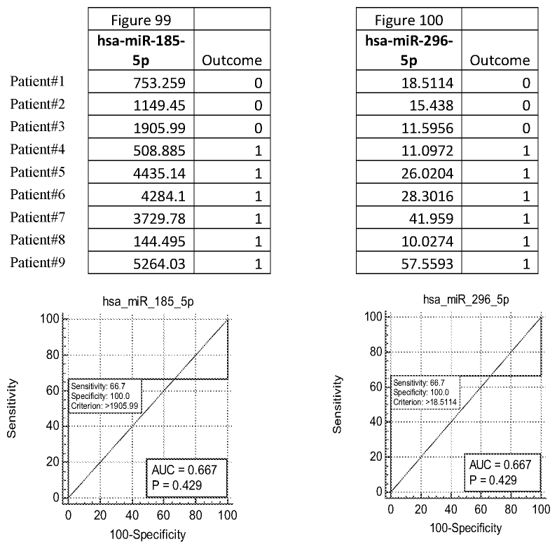

In some embodiments, this disclosure provides methods for identifying a pregnant human being as being at risk for a placental bed disorder, the methods comprising: a) quantifying at least one microRNA (miRNA) from a biological sample derived from immune cells of the pregnant human being; b) identifying the pregnant human being as being at risk for a placental bed disorder on the basis of a difference in the expression of the at least one miRNA as compared to a control biological sample; and, c) optionally treating the pregnant human being identified in step b) as being at risk for a placental bed disorder to ameliorate the likelihood of the occurrence of said placental bed disorder in said pregnant human being, and/or to treat said placental bed disorder in said pregnant human being; wherein: the at least one miRNA is selected from the group consisting of at least one of the miRNAs listed in Table 3, Table 4 or Table 5; and/or at least one of SEQ ID NOS. 1-100; at least any one or more of the miRNAs of FIGS. 1-100; and/or, at least one of hsa-miR-4485-5p (FIG. 19), hsa-miR-551b-3p (FIG. 60), hsa-miR-24-1-5p (FIG. 53), hsa-miR-6819-3p (FIG. 27), hsa-miR-1238-3p (FIG. 14), hsa-miR-6737-3p (FIG. 10), hsa-miR-1237-3p (FIG. 28), hsa-miR-6757-3p (FIG. 40), hsa-miR-6889-3p (FIG. 16), hsa-miR-6752-3p (FIG. 38), hsa-miR-191-3p (FIG. 52), hsa-miR-6795-3p (FIG. 79), hsa-miR-149-5p (FIG. 20), hsa-miR-2116-3p (FIG. 8), hsa-miR-7974 (FIG. 3), hsa-miR-23c (FIG. 24), hsa-miR-4310 (FIG. 9), hsa-miR-98-3p (FIG. 7), hsa-miR-3190-5p (FIG. 5), and/or hsa-miR-4312 (FIG. 39); and/or at least one or more equivalent(s) thereof.

In some embodiments, the methods disclosed here in comprise the steps of: a) quantifying the expression of one or more microRNAs (miRNAs) in a biological sample of a pregnant human being, the miRNAs being: at least one miRNA is selected from the group consisting of at least one of the miRNAs listed in Table 3, Table 4 or Table 5; and/or at least one of SEQ ID NOS. 1-100; and/or, at least one of hsa-miR-4485-5p (FIG. 19), hsa-miR-551b-3p (FIG. 60), hsa-miR-24-1-5p (FIG. 53), hsa-miR-6819-3p (FIG. 27), hsa-miR-1238-3p (FIG. 14), hsa-miR-6737-3p (FIG. 10), hsa-miR-1237-3p (FIG. 28), hsa-miR-6757-3p (FIG. 40), hsa-miR-6889-3p (FIG. 16), hsa-miR-6752-3p (FIG. 38), hsa-miR-191-3p (FIG. 52), hsa-miR-6795-3p (FIG. 79), hsa-miR-149-5p (FIG. 20), hsa-miR-2116-3p (FIG. 8), hsa-miR-7974 (FIG. 3), hsa-miR-23c (FIG. 24), hsa-miR-4310 (FIG. 9), hsa-miR-98-3p (FIG. 7), hsa-miR-3190-5p (FIG. 5), and/or hsa-miR-4312 (FIG. 39); and/or at least one or more equivalent(s) thereof; b) comparing the expression of the miRNAs quantified in step a) to the expression of the same miRNAs in a control sample to determine whether the pregnant human being is at risk of developing preeclampsia, wherein an increase in expression in the pregnant human being relative to the control biological sample indicates the pregnant human being is at risk of developing a placental bed disorder; and, c) optionally treating a pregnant human being identified in step b) as being at risk of developing a placental bed disorder.

Reagents and kits for carrying out such methods are also provided. Other embodiments are also disclosed as will be understood by those of ordinary skill in the art.

### DETAILED DESCRIPTION

This disclosure provides microRNA (miRNA)-based tests and treatment protocols for identifying women at risk for a placental bed disorder (or having a placental bed disorder), also referred to herein as a “compromised pregnancy outcome” (or “compromised” or “compromised outcome”; i.e., as compared to a “healthy pregnancy outcome” (or “healthy” or “healthy outcome”) that does not involve a placental bed disorder). As shown herein, in some embodiments, a ratio (“HC Ratio”) for an individual miRNA can be calculated and used to identify miRNAs of interest. The HC ratio is calculated by using as the numerator the mean miRNA signal (i.e., expression) for a “compromised pregnancy outcome” population minus the mean miRNA signal level (i.e., expression) for a “healthy pregnancy outcome” population (in other words, subtracting the mean miRNA signal level for a “healthy pregnancy outcome” population from the mean miRNA signal for a “compromised pregnancy outcome” population), and using as the denominator the average of the standard deviations (SD) of the “healthy pregnancy outcome” mean signal level and the “compromised pregnancy outcome” mean signal level. Thus, the HC ratio can be calculated as shown below:

$$\frac{\begin{matrix}mean miRNA signal ( compromised ) - \\ mean miRNA signal ( healthy )\end{matrix}}{( S D ( healthy ) + S D ( comprised ) ) / 2}$$

The individual miRNAs identified with high HC ratios are shown herein to distinguish the two populations, for example, those with a placental bed disorder (e.g., preeclampsia) from those women destined to have healthy pregnancy outcome. In a preferred embodiment the HC ratio shall be equal or greater than about any of 1.0, 1.1, 1.2, 1.3, 1.4, or 1.5, and is most preferably equal to or greater than 1.3 (see, e.g., the results presented in Table 3). As shown herein, for microRNAs that demonstrate a high ratio, the “associated criterion value” at the Youden index J point of the ROC calculation can be used to determine the cut-off value used to determine patient risk of developing a placental bed disorder. In some embodiments, when a patient's measured miRNA signal is greater than this predetermined cut-off value, the patient is deemed to be at “higher risk” of experiencing a placental bed disorder. The women in whom a higher miRNA signal is observed can then be further supervised and/or treated, as appropriate, in order to prevent and/or treat placental bed disorders. In some embodiments, such miRNAs can be one or more (i.e., at least one) of the miRNAs listed in Table 3, Table 4 or Table 5; and/or at least one of SEQ ID NOS. 1-100; and/or, at least one of hsa-miR-4485-5p (FIG. 19), hsa-miR-551b-3p (FIG. 60), hsa-miR-24-1-5p (FIG. 53), hsa-miR-6819-3p (FIG. 27), hsa-miR-1238-3p (FIG. 14), hsa-miR-6737-3p (FIG. 10), hsa-miR-1237-3p (FIG. 28), hsa-miR-6757-3p (FIG. 40), hsa-miR-6889-3p (FIG. 16), hsa-miR-6752-3p (FIG. 38), hsa-miR-191-3p (FIG. 52), hsa-miR-6795-3p (FIG. 79), hsa-miR-149-5p (FIG. 20), hsa-miR-2116-3p (FIG. 8), hsa-miR-7974 (FIG. 3), hsa-miR-23c (FIG. 24), hsa-miR-4310 (FIG. 9), hsa-miR-98-3p (FIG. 7), hsa-miR-3190-5p (FIG. 5), and/or hsa-miR-4312 (FIG. 39); and/or at least one or more equivalent(s) thereof.

Within this disclosure, the term “placental bed disorder” refers to conditions that can arise during pregnancy, typically have deleterious effects on and/or during pregnancy, and includes but is not limited to preeclampsia, preterm birth, HELLP Syndrome (a complication of pregnancy characterized by hemolysis, elevated liver enzymes, and a low platelet count), gestational diabetes, miscarriage, implantation failure, intrauterine growth retardation (IUGR) or fetal growth restriction, and premature rupture of the membranes (P.R.O.M.). Within this disclosure, the term “placental site” shall refer to the discrete area of the maternal endometrium in direct contact with the implanting feto-placental unit, which is coextensive with the placenta.

With this disclosure, specific microRNAs may be identified by their prefix mir- and their identifier, such as mir-155. Sequences within an RNA transcript targeted by miRNAs may lie anywhere within the transcript. However, sequences within the 3′ untranslated region are most common. MicroRNA nomenclature comprises a three-letter prefix “mir” followed by a number assigned generally in order of the description of the microRNA. In one convention, when the “R” is lower case, the sequence refers to the pre-microRNA while when upper case is employed (miR), the mature form is indicated. Variants where the sequences vary by one or two bases may be designated by the letters “a” and “b”. Occasionally, pre-microRNAs located within separate regions of the genome result in an identical mature microRNA. These microRNAs are distinguished by a numeric suffix (e.g., “miR-123-1” and “miR-123-2”). When two microRNAs originate from opposite arms of the same pre-microRNA they are designated with the suffix-3p or -5p according to whether the 3′ or 5′ strand is used. As used herein, the numeric code, e.g., “mir-123” shall include its variants such as mir-123-1, mir123-2, and the -3p and -5p variants. As used herein the term “pri-miRNA” shall mean the RNA targeted by the Drosha-Pasha complex; the term “pre-miRNA” shall mean the product of the cleavage by the Drosha-Pasha complex; and, no distinction shall be made between sequences between the parent nomenclature for example mir-123 and any more selective sequence for example mir-123-5p and other than by description within the text. Specific microRNA abbreviations may also include an additional prefix identifying the species of origin, such as “has” for Homo sapiens. miRNAs typically comprise approximately 18-25 nucleotides, in some embodiments, about 22 nucleotides. Nomenclature for miRNAs as used herein may be found in miRBase (www.mirbase.org), the entries of which represent the predicted hairpin portion of the miRNA transcript. It is also noted, as would be understood by those of ordinary skill in the art, that while specific miRNAs are listed in the Tables, Figures and Examples, a number of microRNA equivalents are recognized including, e.g., isomirs (i.e., nongenomic changes made by imprecise cleaving of the microRNA from precursors, 3′ and 5′ additions and deletions), alleles, and the like, and that, in certain embodiments, the methods, reagents and kits of this disclosure comprising such miRNA equivalents are intended to be included therein. Although the primary embodiments described herein are directed to humans, one of skill in the art will appreciate that, in some embodiments, the methods provided in this disclosure can be applied to other species.

As will be discussed below, examples of suitable microRNAs that may be used according to this disclosure include, without limitation, at least one of the miRNAs listed in Table 3, Table 4 or Table 5; at least one of SEQ ID NOS. 1-100; and/or the miRNAs listed in any FIGS. 1-100; and/or, at least one of hsa-miR-4485-5p (FIG. 19), hsa-miR-551b-3p (FIG. 60), hsa-miR-24-1-5p (FIG. 53), hsa-miR-6819-3p (FIG. 27), hsa-miR-1238-3p (FIG. 14), hsa-miR-6737-3p (FIG. 10), hsa-miR-1237-3p (FIG. 28), hsa-miR-6757-3p (FIG. 40), hsa-miR-6889-3p (FIG. 16), hsa-miR-6752-3p (FIG. 38), hsa-miR-191-3p (FIG. 52), hsa-miR-6795-3p (FIG. 79), hsa-miR-149-5p (FIG. 20), hsa-miR-2116-3p (FIG. 8), hsa-miR-7974 (FIG. 3), hsa-miR-23c (FIG. 24), hsa-miR-4310 (FIG. 9), hsa-miR-98-3p (FIG. 7), hsa-miR-3190-5p (FIG. 5), and/or hsa-miR-4312 (FIG. 39); and/or at least one or more equivalent(s) thereof. The methods, reagents and kits disclosed herein may also be as described in U.S. Ser. No. 13/899,555 filed May 21, 2013 (now U.S. Pat. No. 10,323,282 B2 issued on Jun. 8, 2019); PCT/US2012/061994 filed on Oct. 25, 2012; U.S. Ser. No. 13/284,739 filed on Oct. 28, 2011; U.S. Ser. No. 61/767,669 filed on Feb. 21, 2013; and/or U.S. Ser. No. 61/456,063 filed on Nov. 1, 2010; each of which being incorporated herein into this application in their entireties.

Within this disclosure, the term “non-placental biological sample” shall mean maternal cells and derivatives thereof not collected from the placental site such as, e.g., the peripheral blood, of a subject (e.g., a pregnant human being). A non-placental biological sample may be derived from an individual being investigated for the propensity or likelihood of developing a placental bed disorder, or having a placental bed disorder, during the first trimester of a pregnancy, and/or from a control subject. As used herein, the term “subject” refers to any mammal, including both human and other mammals. A “control subject” is an individual(s) of comparable characteristics such as age, sex, and/or condition (e.g., pregnant) who does not have a placental bed disorder, and/or related condition(s) and/or pathology leading to said a placental bed disorder, and are not at known to be at risk of developing a placental bed disorder. The term “control sample” mean a non-placental biological sample of a control subject, taken from the same source, such a peripheral blood, and collected under the same or comparable conditions as a patient sample comprising cells of the non-placental biological sample collected from a control individual that is processed and analyzed in the same manner as a patient sample (e.g., test sample). In some embodiments, the term “control sample” as used herein may represent the mathematical mean of multiple samples from control individuals wherein a number of samples considered sufficient by an individual of ordinary skill in the art are collected. Additional statistical parameters may be derived from said samples such as standard deviation of the mean. Said additional statistical parameters may be used for purposes of comparison of a patient test result with control samples to estimate the probability that the patient's test result represents an abnormal finding and, thereby suggests that the patient is suffering from preeclampsia and related conditions or risk of said condition. For purposes of simplicity the term may also be used in another way wherein a plurality of comparable, temporally separate, samples are collected and assayed from a single individual and compared with one another such that a first sample or a particular subsequent sample are compared as though the first is a control for the second, permitting assessment of a change in condition potentially as a function of the clinical state, or stage of pregnancy or as a result of therapeutic intervention. Preferably, the subjects to whom the methods described herein are applied are human beings, most preferably pregnant human beings.

Suitable techniques for isolating cells from non-placental biological sample can include isopycnic density-gradient centrifugation or monoclonal antibody paramagnetic bead conjugates, for example, as are well-known known in the art as well as any other suitable techniques that are available to those of ordinary skill in the art. In some embodiments, this disclosure provides methods comprising providing a non-placental biological sample. Such a non-placental biological sample can be being derived from cells of the biologic sample such as, for example, peripheral blood (e.g., whole blood), the buffy coat thereof (i.e., the fraction of an anticoagulated peripheral blood sample that contains most of the white blood cells and platelets following density gradient centrifugation of the blood), bone marrow, or other source and then isolating mononuclear cells (e.g., as taught by Boyum (Scand J Immunol 17: 429-436 (1983)). In a preferred embodiment, for example, a sample derived from a peripheral blood and/or bone marrow can include any leukocyte population(s), for example, monocytes, lymphocytes, granulocyte, platelets, and/or stem cells may be segregated by means well known in the art permits selective quantification of miRNAs within that cell population. Further, for example, cell subpopulations (e.g., T cells, B cells) can be individually interrogated following their selective isolation by techniques such as, for example, flow cytometric sorting following interaction with fluorescently labeled monoclonal antibody combinations that are capable of discreetly characterizing the individual subclasses. It is understood by those of ordinary skill in the art that the miRNA content of a sample enriched for mononuclear cells (e.g., the buffy coat) is representative of the miRNA content of the mononuclear cells in that sample because the miRNA content of mononuclear cells is vastly greater than that of plasma. Thus, in preferred embodiments, a buffy coat specimen or even a whole blood specimen is essentially equivalent to a mononuclear cell specimen.

Exemplary methods for isolating RNA include phenol-based extraction and silica matrix or glass fiber filter (GFF)-based binding. Phenol-based reagents comprise various components that denaturants sample constituents, possess the capacity to inhibit RNase's that permit cell and tissue disruption that is followed by steps that permit separation of the RNA from other constituents of the sample. Commercial reagents and kits may be configured to recover short RNA polynucleotides of microRNA length. Extraction procedures such as those using Trizol or TriReagent are useful wherein both long and short RNA polynucleotides are desired. Advantage may be taken of the relative quantity of cell-comprised microRNA versus the quantity of microRNA comprised in the blood liquid phase as in plasma or serum-comprised vesicular structures. The relative quantity of microRNA in the former is very substantially greater than the later permitting assessment of cellular microRNA as a measured by total blood microRNA. The PAXgene blood RNA Tube™ is designed for the collection, storage, stabilization and transport of intracellular RNA, and may be utilized, optionally in conjunction with a nucleic acid purification kit (e.g., the PAXgene Blood RNA Kit) for isolation of cellular miRNA. Isolated cells can be interrogated in batch assays assessing the total quantity of a specific miRNA that may be related to the average quantity expressed by cells of the individual cell type, or may be quantified by in situ hybridization. It is understood herein that detection of miRNA may include detection of the presence or absence of a specific microRNA within a non-placental biological sample, and more preferably its quantification. The methods may produce quantitative or semi-quantitative results. It is understood that relative quantification wherein comparative levels between the sample of the patient is related to the level in a control or other sample particularly wherein sequential samples are assayed. Any detection method well known to those skilled in the art falls within the scope of the invention. Hybridization, preferably where a polynucleotide complimentary to the target polynucleotide is labeled, may be used to detect the target strand. Polymerase chain reaction (PCR) using labeled probes, electrophoresis, and/or sequencing of target strands, or other detection strategy may be employed.

In some embodiments, RNA can be extracted from cells of the non-placental biological sample according to well-known techniques. Blood collected can be drawn into heparinized tubes and maintained at room temperature preferably for approximately 24 hours prior to isolation of cells. RNA sampling and extraction: cells or sorted cell populations (<1×107 viable cells) were collected in 1 ml TRIzol (Invitrogen) and stored at −80° C. until use). Total RNA can be isolated according to standard techniques, such as using the TRIzol reagent/protocol (Invitrogen) and/or RNeasy Mini Kit (Qiagen) (e.g., at room temperature with the QIAcube automated robot (Qiagen)). Total RNA yield can be assessed using the Thermo Scientific NanoDrop 1000 micro-volume spectrophotometer (absorbance at 260 nm and the ratio of 260/280 and 260/230), and RNA integrity assessed using, e.g., the Agilent's Bioanalyzer NANO Lab-on-Chip instrument (Agilent). miRNAs may be quantitated by any suitable technique including but not limited to quantitative real time PCR (qPCR using, e.g., SYBR® Green, a TaqMan® probe, locked nucleic acid probe (Vester, et al. Nature Methods, 7: 687-692 (2004)), miRNA arrays, next generation sequencing (NGS) techniques (e.g., TruSeq kits (Illumina); Baker et al. Biochemistry, 43: 13233-13241 (2010)), multiplex miRNA profiling assays (e.g., FirePlex® miRNA assays), and the like, and/or other available techniques.

In some embodiments, the expression of various miRNAs (e.g., those of Tables 2 and/or Table 3) in a non-placental biological sample of an individual can be collected and assembled to provide a miRNA signature for that individual. Analysis and/or comparison of a microRNA signature of a non-placental biological sample may be compared with a corresponding microRNA signature derived from a control sample and/or a database representive of a control sample. Mathematical approaches to analysis of data and methods for comparison are well known to those skilled in the art and can include, for example, Signal to Noise ratios, Fold Quotients, correlation and statistical methods as hypothesis tests such as t-test, the Wilcoxon-Mann-Whitney test, the Area under the Receiver operator Characteristics Curve Information. Theory approaches, for example, the Mutual Information, Cross-entropy, Probability theory, for example, joint and conditional probabilities can also be appropriate. Combinations and modifications of the previously mentioned examples are understood to be within the scope of the present invention. Heuristic methods may be applied as the database expands.

The methods for quantifying or semi-quantifying microRNA(s) are well-known in the art. These include but are not limited to nucleic acid hybridization techniques well-known in the art for example performed using a solid phase support comprising specific, bound polynucleotides complementary to the target microRNA sequence. RNA isolated from a biologic sample may be reversed transcribed into DNA and conjugated with a detectable label and thence contacted with the anchored probes under hybridizing conditions and scanned by a detection system permitting discrete quantification of signals. It is understood that probe sequences may also be complementary to target sequences comprising SNPs. Moreover, it is understood that probe sequences may be complementary to pre-microRNA and pri-microRNA regions of specific microRNAs. Techniques comprising the polymerase chain reaction (PCR), preferably those incorporating real-time techniques, wherein amplification products are detected through labeled probes or utilizing non-specific dye amplicon-binding dyes such as Cyber Green™. For instance, RNA may be extracted from cells isolated cells by extraction according to instructions from the manufacturer (Qiagen catalogue 763134). microRNA such as for mir-155 may be detected and quantified by polymerase chain reaction (PCR) by the method described by Chen et al. (http://www3.appliedbiosystems.com/cms/groups/mcb_marketing/documents/generaldocuments/cms_040548.pdf downloaded May 11, 2010). Primers and reagents may be selected for individual microRNAs from those described in product overview (http://www3.appliedbiosystems.com/cms/groups/mcb_marketing/documents/generaldocuments/ems_068884.pdf downloaded May 11, 2010).

In some embodiments, an individual identified as being at risk for a placental bed disorder (or as having a placental bed disorder) may be treated by a therapeutic intervention that can prevent, slow, or eliminate the placental bed disorder. Exemplary therapeutic intervention(s) can include any one or more of immunotherapy (e.g., administration of a immunosuppresent and/or anti-inflammatory drug such as intravenous immunoglobulin (IVIG), corticosteroids, Neupogen®), anticoagulant(s) (e.g., heparin(s) such as low molecular weight versions such as Lovenox®), statin(s), progesterone, antibiotic(s), metformin, Cerclage, intralipids, “natural” therapies (e.g., omega-3 and/or fish or krill oil preparations, and the like), dietary changes and/or restrictions, bedrest regimens, and the like. In some embodiments, the appropriate therapeutic intervention can be selected using various in vitro cell markers (e.g., of peripheral blood mononuclear cells (PBMCs)). In some embodiments, quantification of various miRNAs and patterns of miRNA change (e.g., at least one of the miRNAs listed in Table 3, Table 4 or Table 5; at least one of SEQ ID NOS. 1-100; and/or any one or more of the miRNAs of FIGS. 1-100; and/or, at least one of hsa-miR-4485-5p (FIG. 19), hsa-miR-551b-3p (FIG. 60), hsa-miR-24-1-5p (FIG. 53), hsa-miR-6819-3p (FIG. 27), hsa-miR-1238-3p (FIG. 14), hsa-miR-6737-3p (FIG. 10), hsa-miR-1237-3p (FIG. 28), hsa-miR-6757-3p (FIG. 40), hsa-miR-6889-3p (FIG. 16), hsa-miR-6752-3p (FIG. 38), hsa-miR-191-3p (FIG. 52), hsa-miR-6795-3p (FIG. 79), hsa-miR-149-5p (FIG. 20), hsa-miR-2116-3p (FIG. 8), hsa-miR-7974 (FIG. 3), hsa-miR-23c (FIG. 24), hsa-miR-4310 (FIG. 9), hsa-miR-98-3p (FIG. 7), hsa-miR-3190-5p (FIG. 5), and/or hsa-miR-4312 (FIG. 39); and/or at least one or more equivalent(s) thereof) in maternal cells at various time points prior to and following immunotherapeutic intervention may be performed. These miRNA “signatures” can direct the clinical diagnosis and/or treatment. This disclosure also contemplates that the methods, reagents and kits described herein can be used to assess other clinical conditions beyond placental bed disorders and/or different immunotherapeutic interventions. Their use simplifies complex diagnostic strategies into a single procedure and provides information heretofore unavailable. In some embodiments, the methods described herein can include detecting expression of the miRNAs (and/or symptoms of a placental bed disorder) before, during and/or after such therapeutic intervention and treatment can be adjusted according to such expression.

The methods described herein can then comprise quantification of a plurality of individual miRNAs from the non-placental biological sample and quantifying the individual miRNAs and comparing the amount of miRNA(s) in the test sample to the expression of the corresponding microRNA in control sample(s). A significant difference in the amount of miRNA expressed in a test and control samples (i.e., between the test and the control subjects) can indicate the test subject is at risk of developing and/or has a placental bed disorder. In contrast, where there is not a difference (e.g., a significant difference) in the expression of such miRNA(s) between the test and control samples indicates the test subject is not at risk of developing, or does not have, a placental bed disorder. In some embodiments, the method further comprises selecting a treatment or modifying a treatment based on the amount of the one or more RNAs determined. This determination may be based upon assessment of specific individual or combinations of the individual microRNAs. Thus, in some embodiments, this disclosure provides methods for diagnosing a disease or condition, comprising the steps (1) quantifying miRNAs within a predetermined miRNA profile in a non-placental biological sample from an individual (e.g., patient or subject); and (2) comparing said miRNA profile to a reference, wherein the reference is the set of quantifications of said miRNA profile of one or the average of many individuals that are without disease or have a second condition to which the first condition is to be distinguished or compared (e.g., a control sample). This comparison permits diagnosis. Wherein the comparison is between two temporally separate non-placental biological samples of the same individual, it may be used to determine clinical progress. Wherein the two non-placental biological samples of the same individual span a therapeutic intervention, the relative efficacy of therapy may be assessed. Thus, the methods described herein can include the separation of patients into groups distinguishable by characteristic changes in single or multiple microRNAs (e.g., those with or without a risk of development a placental bed disorder), optionally following the selected therapeutic intervention. Identification of patients belonging to microRNA response groups is associated with improved efficacy, prognosis and utility of particular therapeutic intervention(s). Moreover, quantitative levels of certain microRNAs and patterns of change within microRNAs may predict patient response group(s) and post-therapy levels may have additional predictive value. Use of microRNA patterns responsive to therapeutic intervention or predictive thereof provides useful insights into management unavailable through identification of markers directly related to the pathologic process

In some embodiments, expression profiles may consist of the entirety of all known microRNAs incorporated into or onto a microarray chip, bead or other solid support typically used in expression analysis. Any of several methods may be used for quantification or semi-quantification. Determination of an expression profile may be performed by quantitative or semi-quantitative determination of a panel of microRNAs in patients affected by a condition to be assessed and in individuals without said condition. Alternatively, determination of an expression profile that may be used to determine progress of a condition may be determined in a similar manner wherein comparison is made by quantitative or semi-quantitative differences between the two time points. Separate expression profiles may be determined in a similar manner wherein the two time points are separated by a therapeutic intervention. In a similar manner individual expression profiles may be determined at different time points particularly during the course of pregnancy including time points within 6 months preceding or following pregnancy by a term of approximately six months. Panels of miRNAs to be assessed selected a priori or these may comprise large collections intended to include all currently known microRNAs such as in a microarray. The determination may be carried out by any means for determining the expression profiles of nucleic acids (e.g., miRNAs).

In some embodiments, e.g., as described in the Examples, the mean and standard deviation of the expression levels for each miRNA (e.g., those listed in Table 3, Table 4, and/or ffTable 5) from patient samples with “healthy” outcomes and also “compromised” outcomes (e.g., identified as “0” and “1”, respectively, in FIGS. 1-100). To identify miRNAs useful for distinguishing the two populations, a ratio was calculated for each miRNA (the HC ratio), in which where the numerator comprises the absolute difference between the mean value of each of the two populations (“healthy” and “compromised”) and the denominator comprises the average of the two standard deviations of the values for healthy and compromised individuals. In preferred embodiments, one or more miRNAs exhibiting high ratios can be used to differentiate between the two populations of individuals, for example, those individuals with or at risk for developing a placental bed disorder (e.g., preeclampsia; e.g., “1” in FIGS. 1-100) from those individuals destined to have healthy pregnancy outcomes (e.g., “0” in FIGS. 1-100). Table 3 presents the 100 microRNAs exhibiting the highest HC ratios and, in preferred embodiments, can be used to differentiate those individuals with or at risk for developing a placental bed disorder (e.g., preeclampsia; e.g., “1” in FIGS. 1-100) from those destined to have healthy pregnancy outcomes (e.g., “0” in FIGS. 1-100). The data generated for each miRNA can also be subjected to a Receiver Operating Characteristics (ROC) curve analysis generating area under the curve (AUC) data with each miRNA's respective p values. In some embodiments (as in the Examples here), the data from the 100 miRNAs exhibiting the highest HC ratios (Table 3) can be subjected to ROC curve analysis. In Table 4, the miRNAs are presented in order of highest HC ratio. In Table 5, microRNAs are listing by their Clinical Value Ranking. The 100 microRNAs that were originally selected by HC Ratio, are further selected for clinical utility based on additional selection criteria (1) adequate signal strength >5.0, (2) signal consistency (>85% of patients demonstrate signal) and (3) ROC curve p value <0.05. As seen in Table 5, an “x” designates a microRNA that fulfils selection criteria designated at top of the respective column. Twenty microRNA fulfil all selection criteria. Individual ROC curve calculations on the nine patient samples described in Table 1 are shown in FIGS. 1-100 for the 50 miRNAs with the highest ratios. (listed in the same order as the HC ratio ranking in Table 4). The p value indicates the reliability of the individual microRNAs, and lower p values indicate microRNAs with higher predictive power. As shown in Table 5, these 20 miRNAs are hsa-miR-4485-5p (FIG. 19), hsa-miR-551b-3p (FIG. 60), hsa-miR-24-1-5p (FIG. 53), hsa-miR-6819-3p (FIG. 27), hsa-miR-1238-3p (FIG. 14), hsa-miR-6737-3p (FIG. 10), hsa-miR-1237-3p (FIG. 28), hsa-miR-6757-3p (FIG. 40), hsa-miR-6889-3p (FIG. 16), hsa-miR-6752-3p (FIG. 38), hsa-miR-191-3p (FIG. 52), hsa-miR-6795-3p (FIG. 79), hsa-miR-149-5p (FIG. 20), hsa-miR-2116-3p (FIG. 8), hsa-miR-7974 (FIG. 3), hsa-miR-23c (FIG. 24), hsa-miR-4310 (FIG. 9), hsa-miR-98-3p (FIG. 7), hsa-miR-3190-5p (FIG. 5), and hsa-miR-4312 (FIG. 39). Individual ROC curve calculations on the nine patient samples described in Table 1 are shown in FIGS. 1-100 for the 100 miRNAs with the highest ratios (i.e., those listed in Table 4). In some embodiments, the miRNAs identified by such methods that can be used in the methods for distinguishing individuals with or at risk for a placental bed disorder (e.g., “1” in FIGS. 1-100) from those individuals not having or being at risk for a placental bed disorder (e.g., “0”). Other methods for determining miRNAs suitable for use in the methods may also be used. In some embodiments, suitable miRNAs for use in the methods described herein for distinguishing individuals with or at risk for a placental bed disorder from those individuals not having or being at risk for a placental bed disorder may have the ratio, AUC, 95% Confidence Interval, p value, Youden index J, the sensitivity, specificity, and/or criterion of any of the miRNAs described in Table 4 and/or illustrated in any one or more of FIGS. 1-100. These techniques were utilized to identify miRNAs indicative of a placental bed disorder as shown in FIGS. 1-100. As shown therein, a significant different in the miRNA signal levels for certain miRNAs was observed between women who experienced a healthy delivery and those who did not (“0” and “1” in FIGS. 1-100, respectively). In some embodiments, by this method of analysis, these miRNAs include at least one of the miRNAs listed in Table 3, Table 4 or Table 5; least one of SEQ ID NOS. 1-100; and/or any one or more miRNAs listed in FIGS. 1-100; and/or, at least one of hsa-miR-4485-5p (FIG. 19), hsa-miR-551b-3p (FIG. 60), hsa-miR-24-1-5p (FIG. 53), hsa-miR-6819-3p (FIG. 27), hsa-miR-1238-3p (FIG. 14), hsa-miR-6737-3p (FIG. 10), hsa-miR-1237-3p (FIG. 28), hsa-miR-6757-3p (FIG. 40), hsa-miR-6889-3p (FIG. 16), hsa-miR-6752-3p (FIG. 38), hsa-miR-191-3p (FIG. 52), hsa-miR-6795-3p (FIG. 79), hsa-miR-149-5p (FIG. 20), hsa-miR-2116-3p (FIG. 8), hsa-miR-7974 (FIG. 3), hsa-miR-23c (FIG. 24), hsa-miR-4310 (FIG. 9), hsa-miR-98-3p (FIG. 7), hsa-miR-3190-5p (FIG. 5), and/or hsa-miR-4312 (FIG. 39); and/or at least one or more equivalent(s) thereof. In some embodiments, at least any of one, two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 of SEQ ID NOS. 1-100, or equivalents thereof, may be used in the methods, reagents and/or kits disclosed herein. In some embodiments, the miRNAs utilized can exhibit signal consistency of greater than about 85% in patients, exhibit a mean signal strength of greater than about 5.0, and be significant with a p<0.05 (e.g., as shown for the 20 miRNAs ranked as 1-20 in Table 5 (i.e., hsa-miR-4485-5p, hsa-miR-551b-3p, hsa-miR-24-1-5p, hsa-miR-6819-3p, hsa-miR-1238-3p, hsa-miR-6737-3p, hsa-miR-1237-3p, hsa-miR-6757-3p, hsa-miR-6889-3p, hsa-miR-6752-3p, hsa-miR-191-3p, hsa-miR-6795-3p, hsa-miR-149-5p, hsa-miR-2116-3p, hsa-miR-7974, hsa-miR-23c, hsa-miR-4310, hsa-miR-98-3p, hsa-miR-3190-5p, and/or hsa-miR-4312)). Other miRNAs may also be useful, as may be determined by those of ordinary skill in the art.

Thus, in some embodiments, this disclosure provides reagents and methods for identifying at least two characteristic groups in a patient population on the basis of microRNA (miRNA) expression, wherein one characteristic group is associated with a reproductive disorder or a risk of developing such a disorder, comprising the steps of: a) quantifying at least one microRNA from a biological sample derived from immune cells; and, b) segregating the patient population into the groups on the basis of expression of the at least one miRNA, wherein: the miRNA is selected from the group consisting of at least one the miRNAs listed in Table 3, Table 4, Table 5, SEQ ID NOS. 1-100, and/or the miRNAs referred to in FIGS. 1-100; and/or, the at least one miRNA is selected from the group consisting of hsa-miR-4485-5p, hsa-miR-551b-3p, hsa-miR-24-1-5p, hsa-miR-6819-3p, hsa-miR-1238-3p, hsa-miR-6737-3p, hsa-miR-1237-3p, hsa-miR-6757-3p, hsa-miR-6889-3p, hsa-miR-6752-3p, hsa-miR-191-3p, hsa-miR-6795-3p, hsa-miR-149-5p, hsa-miR-2116-3p, hsa-miR-7974, hsa-miR-23c, hsa-miR-4310, hsa-miR-98-3p, hsa-miR-3190-5p, and/or hsa-miR-4312. In some embodiments, the step of segregating the patient population comprises assigning patients expressing a relatively high level of the at least one miRNA to a first group and assigning patients expressing a relatively low level of the at least one miRNA to a second group. In some such embodiments, the patient population is pregnant human beings and the population segregated in step b) is at risk of developing a placental bed disorder. Such methods may include, in some embodiments, the step of calculating the HC ratio and selecting miRNAs of interest on that basis.

7. In some embodiments, this disclosure provides reagents and methods for identifying a pregnant human being as being at risk for a placental bed disorder, the method comprising: a) quantifying at least one microRNA (miRNA) from a biological sample derived from immune cells of the pregnant human being; b) identifying the pregnant human being as being at risk for a placental bed disorder on the basis of a difference in the expression of the at least one miRNA as compared to a control biological sample; and, c) optionally treating the pregnant human being identified in step b) as being at risk for a placental bed disorder to ameliorate the likelihood of the occurrence of said placental bed disorder in said pregnant human being, and/or to treat said placental bed disorder in said pregnant human being; wherein the at least one miRNA is selected from the group consisting of at least one of the miRNAs listed in Table 3, Table 4 or Table 5; at least one of SEQ ID NOS. 1-100; and/or at least one of the miRNAs referred to in FIGS. 1-100; and/or, at least one of hsa-miR-4485-5p (FIG. 19), hsa-miR-551b-3p (FIG. 60), hsa-miR-24-1-5p (FIG. 53), hsa-miR-6819-3p (FIG. 27), hsa-miR-1238-3p (FIG. 14), hsa-miR-6737-3p (FIG. 10), hsa-miR-1237-3p (FIG. 28), hsa-miR-6757-3p (FIG. 40), hsa-miR-6889-3p (FIG. 16), hsa-miR-6752-3p (FIG. 38), hsa-miR-191-3p (FIG. 52), hsa-miR-6795-3p (FIG. 79), hsa-miR-149-5p (FIG. 20), hsa-miR-2116-3p (FIG. 8), hsa-miR-7974 (FIG. 3), hsa-miR-23c (FIG. 24), hsa-miR-4310 (FIG. 9), hsa-miR-98-3p (FIG. 7), hsa-miR-3190-5p (FIG. 5), and/or hsa-miR-4312 (FIG. 39); and/or at least one or more equivalent(s) thereof. In some embodiments, this disclosure provides methods comprising the steps of: a) quantifying the expression of one or more microRNAs (miRNAs) in a biological sample of a pregnant human being, the miRNAs being: at least one miRNA is selected from the group consisting of at least one of the miRNAs listed in Table 3, Table 4 or Table 5; at least one of SEQ ID NOS. 1-100; at least one of the miRNAs referred to in FIGS. 1-100; and/or, at least one of hsa-miR-4485-5p (FIG. 19), hsa-miR-551b-3p (FIG. 60), hsa-miR-24-1-5p (FIG. 53), hsa-miR-6819-3p (FIG. 27), hsa-miR-1238-3p (FIG. 14), hsa-miR-6737-3p (FIG. 10), hsa-miR-1237-3p (FIG. 28), hsa-miR-6757-3p (FIG. 40), hsa-miR-6889-3p (FIG. 16), hsa-miR-6752-3p (FIG. 38), hsa-miR-191-3p (FIG. 52), hsa-miR-6795-3p (FIG. 79), hsa-miR-149-5p (FIG. 20), hsa-miR-2116-3p (FIG. 8), hsa-miR-7974 (FIG. 3), hsa-miR-23c (FIG. 24), hsa-miR-4310 (FIG. 9), hsa-miR-98-3p (FIG. 7), hsa-miR-3190-5p (FIG. 5), and/or hsa-miR-4312 (FIG. 39); and/or at least one or more equivalent(s) thereof; b) comparing the expression of the miRNAs quantified in step a) to the expression of the same miRNAs in a control sample to determine whether the pregnant human being is at risk of developing preeclampsia, wherein an increase in expression in the pregnant human being relative to the control biological sample indicates the pregnant human being is at risk of developing a placental bed disorder; and, c) optionally treating a pregnant human being identified in step b) as being at risk of developing a placental bed disorder. In some embodiments of such methods, the biological sample (e.g., a blood sample, a peripheral blood sample, bone marrow sample, such as on comprising one or more maternal blood cells such as mononuclear cells) are obtained during the first trimester of pregnancy; the placental bed disorder is selected from the group consisting of preeclampsia, preterm birth, HELLP Syndrome, gestational diabetes, miscarriage, implantation failure, fetal growth restriction, and premature rupture of the membranes (P.R.O.M.). In preferred embodiments, the control biological sample is and/or is representative of a pregnant human being without a placental bed disorder. In some such methods, the step(s) of isolating blood cells such as mononuclear cells from the biological sample, and/or extracting miRNA-comprising RNA from the biological sample are also included. In preferred embodiments, this disclosure provides methods for identifying at least one miRNA that distinguishes a first population individuals at risk for a placental bed disorder from at least one second population comprising individuals not at risk for a placental bed disorder, the method comprising calculating a ratio (i.e., the HC ratio) of expression of said at least one miRNA, wherein said ratio comprises: a numerator equal to the difference between the mean value of expression of the at least one miRNA in the first population minus the mean value of the second population and the denominator comprises the average of the two standard deviations of the values for the first and second populations. In some embodiments, the HC ratio for an individual miRNA can be based on the expression of one or more miRNAs (e.g., at least one of the miRNAs listed in Table 3, Table 4 or Table 5; at least one of SEQ ID NOS. 1-100; at least one of the miRNAs referred to in FIGS. 1-100; and/or, at least one of hsa-miR-4485-5p (FIG. 19), hsa-miR-551b-3p (FIG. 60), hsa-miR-24-1-5p (FIG. 53), hsa-miR-6819-3p (FIG. 27), hsa-miR-1238-3p (FIG. 14), hsa-miR-6737-3p (FIG. 10), hsa-miR-1237-3p (FIG. 28), hsa-miR-6757-3p (FIG. 40), hsa-miR-6889-3p (FIG. 16), hsa-miR-6752-3p (FIG. 38), hsa-miR-191-3p (FIG. 52), hsa-miR-6795-3p (FIG. 79), hsa-miR-149-5p (FIG. 20), hsa-miR-2116-3p (FIG. 8), hsa-miR-7974 (FIG. 3), hsa-miR-23c (FIG. 24), hsa-miR-4310 (FIG. 9), hsa-miR-98-3p (FIG. 7), hsa-miR-3190-5p (FIG. 5), and/or hsa-miR-4312 (FIG. 39); and/or at least one or more equivalent(s) thereof) in immune cells (e.g., peripheral blood, buffy coat) of pregnant women. In preferred embodiments, the numerator of the HC ratio is calculated by subtracting the mean miRNA signal level for a healthy pregnancy outcome population from the mean miRNA signal for a compromised pregnancy outcome population, and the denominator calculated as the average of the standard deviations of the healthy outcome miRNA mean signal and the compromised outcome mean miRNA signal level. The individual miRNAs identified with high HC ratios are shown herein to distinguish the two populations, for example, those with a placental bed disorder (e.g., preeclampsia) from those likely to have a healthy pregnancy outcome. In some embodiments, the at least one miRNA is one exhibiting a HC ratio of greater than or equal to about any of 1.0, 1.1, 1.2, 1.3, 1.4, or 1.5. In preferred embodiments, the HC ratio is equal to or greater than 1.3 (see, e.g., Table 3). In some embodiments, such miRNA exhibits a signal consistency of at least about 85%; a mean signal strength of at least 3.0, 4.0, or preferably 5.0 Ct (PCR cycle threshold); and a p value of less than 0.05 (p<0.05). method for identifying at least one miRNA that distinguishes a first population individuals at risk for a placental bed disorder from at least one second population comprising individuals not at risk for a placental bed disorder, the method comprising calculating the ratio HC ratio, wherein the first population are compromised pregnancy outcome individuals and the second population is healthy pregnancy outcome individuals. In some embodiments, said miRNA exhibits a signal consistency of at least about 85%; a mean signal strength of at least 5.0; and a p value of less than 0.05 (p<0.05).

In some embodiments, this disclosure provides one or more component(s) of a diagnostic assay comprising at least one of the miRNAs listed in Table 3, Table 4 or Table 5; at least one of SEQ ID NOS. 1-100; at least one of the miRNAs referred to in FIGS. 1-100 and/or, at least one of hsa-miR-4485-5p (FIG. 19), hsa-miR-551b-3p (FIG. 60), hsa-miR-24-1-5p (FIG. 53), hsa-miR-6819-3p (FIG. 27), hsa-miR-1238-3p (FIG. 14), hsa-miR-6737-3p (FIG. 10), hsa-miR-1237-3p (FIG. 28), hsa-miR-6757-3p (FIG. 40), hsa-miR-6889-3p (FIG. 16), hsa-miR-6752-3p (FIG. 38), hsa-miR-191-3p (FIG. 52), hsa-miR-6795-3p (FIG. 79), hsa-miR-149-5p (FIG. 20), hsa-miR-2116-3p (FIG. 8), hsa-miR-7974 (FIG. 3), hsa-miR-23c (FIG. 24), hsa-miR-4310 (FIG. 9), hsa-miR-98-3p (FIG. 7), hsa-miR-3190-5p (FIG. 5), and/or hsa-miR-4312 (FIG. 39); and/or at least one or more equivalent(s) thereof; and/or a binding partner (e.g., detection reagent) for at least one of said miRNAs. In some embodiments, the one or more components can be selected from the group consisting of a nucleic acid amplification primer, a pair of nucleic acid amplification primers, and an oligonucleotide probe corresponding to at least one of said miRNAs (“corresponding to” meaning that the component can be used to identify at least one of said miRNAs from a sample, such as a biological sample, using an miRNA detection assay). In some embodiments, this disclosure provides a microarray, solid support, or collection of solid supports, comprising at least one of the miRNAs listed in Table 3, Table 4, Table 5, or FIGS. 1-100; at least one of SEQ ID NOS. 1-100; and/or, at least one of hsa-miR-4485-5p (FIG. 19), hsa-miR-551b-3p (FIG. 60), hsa-miR-24-1-5p (FIG. 53), hsa-miR-6819-3p (FIG. 27), hsa-miR-1238-3p (FIG. 14), hsa-miR-6737-3p (FIG. 10), hsa-miR-1237-3p (FIG. 28), hsa-miR-6757-3p (FIG. 40), hsa-miR-6889-3p (FIG. 16), hsa-miR-6752-3p (FIG. 38), hsa-miR-191-3p (FIG. 52), hsa-miR-6795-3p (FIG. 79), hsa-miR-149-5p (FIG. 20), hsa-miR-2116-3p (FIG. 8), hsa-miR-7974 (FIG. 3), hsa-miR-23c (FIG. 24), hsa-miR-4310 (FIG. 9), hsa-miR-98-3p (FIG. 7), hsa-miR-3190-5p (FIG. 5), and/or hsa-miR-4312 (FIG. 39); and/or at least one or more equivalent(s) thereof; and/or a binding partner for (e.g., a hybridizing nucleic acid) at least one of said miRNAs. In some embodiments, this disclosure provides microarrays, solid supports, or collection of solid supports comprising a nucleic acid amplification primer, a pair of nucleic acid amplification primers, and/or an oligonucleotide probe corresponding to at least one of said miRNAs In some embodiments, the component, microarray, solid support, or collection of solid supports comprise SEQ ID NOS. 1-100; and/or, hsa-miR-4485-5p, hsa-miR-551b-3p, hsa-miR-24-1-5p, hsa-miR-6819-3p, hsa-miR-1238-3p, hsa-miR-6737-3p, hsa-miR-1237-3p, hsa-miR-6757-3p, hsa-miR-6889-3p, hsa-miR-6752-3p, hsa-miR-191-3p, hsa-miR-6795-3p, hsa-miR-149-5p, hsa-miR-2116-3p, hsa-miR-7974, hsa-miR-23c, hsa-miR-4310, hsa-miR-98-3p, hsa-miR-3190-5p, and hsa-miR-4312; and/or a binding partner for at least one of said miRNAs. In some embodiments, the solid support or collection of solid supports is a bead or collection of beads, respectively. In some embodiments, this disclosure provides a kit comprising any such component, microarray, solid support, or collection of solid supports optionally further including instructions for use. Other embodiments are also contemplated, as would be understood by those of ordinary skill in the art.

Within this disclosure, the terms “about”, “approximately”, and the like, when preceding a list of numerical values or range, refer to each individual value in the list or range independently as if each individual value in the list or range was immediately preceded by that term. The terms mean that the values to which the same refer are exactly, close to, or similar thereto. Optional or optionally means that the subsequently described event or circumstance can or cannot occur, and that the description includes instances where the event or circumstance occurs and instances where it does not. Ranges may be expressed herein as from about one particular value, and/or to about another particular value. When such a range is expressed, another aspect includes from the one particular value and/or to the other particular value. Similarly, when values are expressed as approximations, by use of the antecedent about or approximately, it will be understood that the particular value forms another aspect. It will be further understood that the endpoints of each of the ranges are significant both in relation to the other endpoint, and independently of the other endpoint. Ranges (e.g., 90-100%) are meant to include the range per se as well as each independent value within the range as if each value was individually listed.

All references cited within this disclosure are hereby incorporated by reference in their entirety. Certain embodiments are further described in the following examples. These embodiments are provided as examples only and are not intended to limit the scope of the claims in any way.

### EXAMPLES

### Example 1

### Materials and Methods

This study was performed to identify individual microRNAs (miRNAs) isolated from maternal peripheral blood cells that can be used to distinguishing women destined to healthy pregnancies from women more likely to develop a placental bed disorder (e.g., preeclampsia). To enhance the number of patient samples derived from women who ultimately develop a placental bed disorder, a higher risk group (overweight (BMI≥25), black women) was selected from the sample collection for these studies. “Normal delivery” was defined as the delivery of a singleton, normal karyotype baby with the following pregnancy criteria: delivery at 38-42 weeks gestation, baby weight within the normal range for gestational age. Preeclampsia was defined according to the guidelines of the International Society for the Study of Hypertension in Pregnancy (Brown, et al. The classification and diagnosis of the hypertensive disorders of pregnancy: statement from the international society for the study of hypertension in pregnancy (ISSHP). Hypertens Pregnancy 2001). The study was a retrospective analysis using clinical data from patient charts and specimens frozen and stored as buffy coat.

All samples were obtained from a larger collection of patients that met the following criteria: 1) index cycle between April 2006 to July 2014; 2) singleton delivery; 3) delivered baby with no obvious birth defects; and, 4) blood sample available from 11-13 weeks gestation. Venous blood was obtained from women who had given written informed consent to provide samples for research as approved by the National Research Ethics Service of the National Health Service. Blood was collected in EDTA Vacutainer tubes (Becton Dickinson UK Ltd, Oxfordshire, United Kingdom) and processed within 15 minutes of collection. The tubes were centrifuged at 2000 g for 10 minutes. Buffy coat was collected by pipette aspiration guided visually and frozen immediately at −80° C. without an RNA preservative, labeled with a unique patient identifier and maintained for up to nine years. Specimens were shipped from King's College London (KCL), UK directly to the Stanford Human Immune Monitoring Center (Stanford, Calif., USA) on dry ice where they remained blinded as to clinical outcome through testing, identified only by a unique identification number.

Blood samples taken from nine pregnant women in their first trimester of pregnancy was retrospectively evaluated (three healthy women who developed healthy, full term deliveries and six women who developed one or more placental bed disorders, designated “compromised” (Table 2). MicroRNA was isolated according to the procedure given in said paper (Winger, et al. Peripheral blood cell microRNA quantification during the first trimester predicts preeclampsia: Proof of concept. PLoS One. 2018 Jan. 2; 13(1):e0190654), and then subsequently quantified by microarray quantification according to the manufacturer's direction (Human miRNA Microarray, Release 21.0, 8×60K, G4872A-07015 (Agilent Technologies) following labeling performed using miRNA Complete Labeling and Hyb Kit 5190-0456 (Agilent Technologies)). A total of 2,550 microRNAs were interrogated.

Means and standard deviations were calculated for each microRNA from patient samples with “healthy” outcomes and also “compromised” outcomes. To identify individual miRNAs useful for distinguishing the two populations, a “ratio” (“HC Ratio”) was calculated for each miRNA where the numerator comprises the difference between the mean value of the “compromised” population minus the mean value of the “healthy” population and the denominator comprises the average of the two standard deviations of the values for healthy and compromised individuals. The individual miRNAs identified with high ratios (>1.3) are shown herein to discriminate between the two populations. The individual microRNAs identified with high ratios can be employed to discriminate between the two populations. To determine individual patient risk, the ROC curve's associated criterion value (cut-off point”) taken at the Youden J point can be used (as seen in Table 4). When the patient's microRNA signal level is above the cut-off point set at the Youden J point, the patient is deemed to be at “increased risk” of a developing a pregnancy disorder. The Youden J point is determined from ROC curve analysis using Medcalc® software (MedCalc Statistical Software version 18.10.2 (MedCalc Software bvba, Ostend, Belgium; http://www.medcalc.org; 2018) upon analysis of quantification of individual microRNA in patients developing healthy and compromised pregnancies. It is also understood that a plurality of microRNAs could be simultaneously analyzed to enhance predictive power. Of the 2,550 microRNAs that were interrogated in the patient population, the 100 microRNAs with the highest ratios (see the column labeled “HC Ratio”), i.e., those most useful for differentiating patients likely to experience a healthy outcome from those likely to experience a compromised outcome, are presented in Table 4. By this method of analysis, the miRNAs listed in Table 4 could be useful in differentiating between a woman predisposed to a healthy pregnancy outcome from one likely to experience a placental bed disorder.

The 100 miRNAs exhibiting the highest ratios (i.e., those listed in Table 4) were then subjected to a ROC curve analysis generating area under the curve (AUC) with their respective p values. From this, the clinical cut-offs were derived from the ROC statistics (Table 4). Individual ROC curve calculations on the nine patient samples described in Table 2 are shown in FIGS. 1-100 for microRNAs with the highest ratios. The p value indicates the reliability of the individual microRNAs and further refines the microRNA selection process. By this method of analysis, the miRNAs with the lowest p values are even more useful in differentiating between a woman predisposed to a healthy pregnancy outcome from one likely to experience a placental bed disorder

As shown in Table 5, 20 microRNAs can be even further selected for clinical utility based on having a mean signal strength greater than 5.0 Ct signal units (more practical in a clinical setting), a microRNA demonstrating signal consistency (85% of patient samples demonstrate a measurable signal) as well as it's calculated ROC p value being less than or equal to 0.05. By using these additional selection criteria, 20 microRNAs from the original 100 were selected as being most clinically useful (Table 5), and therefore preferred. These miRNAs include hsa-miR-4485-5p (FIG. 19), hsa-miR-551b-3p (FIG. 60), hsa-miR-24-1-5p (FIG. 53), hsa-miR-6819-3p (FIG. 27), hsa-miR-1238-3p (FIG. 14), hsa-miR-6737-3p (FIG. 10), hsa-miR-1237-3p (FIG. 28), hsa-miR-6757-3p (FIG. 40), hsa-miR-6889-3p (FIG. 16), hsa-miR-6752-3p (FIG. 38), hsa-miR-191-3p (FIG. 52), hsa-miR-6795-3p (FIG. 79), hsa-miR-149-5p (FIG. 20), hsa-miR-2116-3p (FIG. 8), hsa-miR-7974 (FIG. 3), hsa-miR-23c (FIG. 24), hsa-miR-4310 (FIG. 9),hsa-miR-98-3p (FIG. 7), hsa-miR-3190-5p (FIG. 5) and/or hsa-miR-4312 (FIG. 39).

While certain embodiments have been described in terms of the preferred embodiments, it is understood that variations and modifications will occur to those skilled in the art. Therefore, it is intended that the appended claims cover all such equivalent variations that come within the scope of the following claims.

What is claimed is:

1. A method for identifying at least two characteristic groups in a patient population on the basis of microRNA (miRNA) expression, wherein one characteristic group is associated with a reproductive disorder or a risk of developing such a disorder, comprising the steps of: a) quantifying at least one microRNA from a biological sample derived from immune cells; and, b) segregating the patient population into the groups on the basis of expression of the at least one miRNA, wherein:
the miRNA is selected from the group consisting of at least one the miRNAs listed in Table 3, Table 4, Table 5, and/or SEQ ID NOS. 1-100; and/or,
the at least one miRNA is selected from the group consisting of hsa-miR-4485-5p, hsa-miR-551b-3p, hsa-miR-24-1-5p, hsa-miR-6819-3p, hsa-miR-1238-3p, hsa-miR-6737-3p, hsa-miR-1237-3p, hsa-miR-6757-3p, hsa-miR-6889-3p, hsa-miR-6752-3p, hsa-miR-191-3p, hsa-miR-6795-3p, hsa-miR-149-5p, hsa-miR-2116-3p, hsa-miR-7974, hsa-miR-23c, hsa-miR-4310, hsa-miR-98-3p, hsa-miR-3190-5p, and/or hsa-miR-4312.

2. The method of claim 1 wherein the step of segregating the patient population comprises assigning patients expressing a relatively high level of the at least one miRNA to a first group and assigning patients expressing a relatively low level of the at least one miRNA to a second group.

3. The method of claim 1 or 2 wherein the patient population is pregnant human beings and the population segregated in step b) is at risk of developing a placental bed disorder.

4. A method for identifying a pregnant human being as being at risk for a placental bed disorder, the method comprising:
a) quantifying at least one microRNA (miRNA) from a biological sample derived from immune cells of the pregnant human being;
b) identifying the pregnant human being as being at risk for a placental bed disorder on the basis of a difference in the expression of the at least one miRNA as compared to a control biological sample; and,
c) optionally treating the pregnant human being identified in step b) as being at risk for a placental bed disorder to ameliorate the likelihood of the occurrence of said placental bed disorder in said pregnant human being, and/or to treat said placental bed disorder in said pregnant human being;
wherein:
the at least one miRNA is selected from the group consisting of at least one the miRNAs listed in Table 3, Table 4, Table 5, and/or SEQ ID NOS. 1-100; and/or,
the at least one miRNA is selected from the group consisting of hsa-miR-4485-5p, hsa-miR-551b-3p, hsa-miR-24-1-5p, hsa-miR-6819-3p, hsa-miR-1238-3p, hsa-miR-6737-3p, hsa-miR-1237-3p, hsa-miR-6757-3p, hsa-miR-6889-3p, hsa-miR-6752-3p, hsa-miR-191-3p, hsa-miR-6795-3p, hsa-miR-149-5p, hsa-miR-2116-3p, hsa-miR-7974, hsa-miR-23c, hsa-miR-4310, hsa-miR-98-3p, hsa-miR-3190-5p, and/or hsa-miR-4312.

5. A method comprising the steps of:
a) quantifying the expression of one or more microRNAs (miRNAs) in a biological sample of a pregnant human being, the miRNAs being:
at least one miRNA is selected from the group consisting of at least one miRNAs listed in Table 3, Table 4, Table 5, and/or SEQ ID NOS. 1-100; and/or,
the at least one miRNA is selected from the group consisting of hsa-miR-4485-5p, hsa-miR-551b-3p, hsa-miR-24-1-5p, hsa-miR-6819-3p, hsa-miR-1238-3p, hsa-miR-6737-3p, hsa-miR-1237-3p, hsa-miR-6757-3p, hsa-miR-6889-3p, hsa-miR-6752-3p, hsa-miR-191-3p, hsa-miR-6795-3p, hsa-miR-149-5p, hsa-miR-2116-3p, hsa-miR-7974, hsa-miR-23c, hsa-miR-4310, hsa-miR-98-3p, hsa-miR-3190-5p, and/or hsa-miR-4312;
b) comparing the expression of the miRNAs quantified in step a) to the expression of the same miRNAs in a control sample to determine whether the pregnant human being is at risk of developing preeclampsia, wherein an increase in expression in the pregnant human being relative to the control biological sample indicates the pregnant human being is at risk of developing a placental bed disorder; and,
c) optionally treating a pregnant human being identified in step b) as being at risk of developing a placental bed disorder.

6. The method of any preceding claim wherein the biological sample is obtained during the first trimester of pregnancy.

8. The method of any preceding claim wherein the placental bed disorder is selected from the group consisting of preeclampsia, preterm birth, HELLP Syndrome, gestational diabetes, miscarriage, implantation failure, fetal growth restriction, and premature rupture of the membranes (P.R.O.M.).

9. The method of any preceding claim, wherein the placental bed disorder is preeclampsia.

10. The method of any preceding claim wherein the control biological sample is representative of a pregnant human being without a placental bed disorder.

11. The method of any preceding claim wherein the biological sample comprises mononuclear cells.

12. The method of any preceding claim wherein the biological sample is peripheral blood.

13. The method of any preceding claim, further comprising the additional step of isolating mononuclear cells from the biological sample.

14. The method of any preceding claim wherein the biological sample is derived from peripheral blood.

15. The method of any preceding claim, further comprising the step of extracting miRNA-comprising RNA from the biological sample.

16. A method of any preceding claim further comprising the steps of quantifying at least one microRNA from a biological sample derived from immune cells from an additional pregnant and identifying the additional pregnant human being as being at risk for a placental bed disorder on the basis of expression of the at least one microRNA.

17. A method for identifying at least one miRNA that distinguishes a first population individuals at risk for a placental bed disorder from at least one second population comprising individuals not at risk for a placental bed disorder, the method comprising calculating a ratio (HC ratio) of expression of said at least one miRNA, wherein said ratio comprises: a numerator equal to the difference between the mean value of expression of the at least one miRNA in the first population and the mean value of the second population and the denominator comprises the average of the two standard deviations of the values for the first and second populations.

18. The method of claim 16 wherein the first population are compromised pregnancy outcome individuals and the second population is healthy pregnancy outcome individuals.

19. The method of claim 16 or 17 wherein said miRNA exhibits a signal consistency of at least about 85%; a mean signal strength of at least 5.0; and a p value of less than 0.05 (p<0.05).

20. The method of any one of claims 16-18 wherein the at least one miRNA exhibits a HC ratio of greater than or equal to about any of 1.0. 1.1, 1.2, 1.3, 1.4, or 1.5; or greater than or equal to about 1.3.

21. A component of a diagnostic assay comprising at least one miRNA listed in Table 3, Table 4, Table 5, and/or SEQ ID NOS. 1-100; and/or, at least one of hsa-miR-4485-5p, hsa-miR-551b-3p, hsa-miR-24-1-5p, hsa-miR-6819-3p, hsa-miR-1238-3p, hsa-miR-6737-3p, hsa-miR-1237-3p, hsa-miR-6757-3p, hsa-miR-6889-3p, hsa-miR-6752-3p, hsa-miR-191-3p, hsa-miR-6795-3p, hsa-miR-149-5p, hsa-miR-2116-3p, hsa-miR-7974, hsa-miR-23c, hsa-miR-4310, hsa-miR-98-3p, hsa-miR-3190-5p, and/or hsa-miR-4312.

22. The component of claim 20 wherein said component is selected from the group consisting of a nucleic acid amplification primer, a pair of nucleic acid amplification primers, and an oligonucleotide probe corresponding to at least one of said miRNAs.

23. A microarray, solid support, or collection of solid supports, comprising at least one miRNA listed in Table 3, Table 4, Table 5, and/or SEQ ID NOS. 1-100; and/or, at least one of hsa-miR-4485-5p, hsa-miR-551b-3p, hsa-miR-24-1-5p, hsa-miR-6819-3p, hsa-miR-1238-3p, hsa-miR-6737-3p, hsa-miR-1237-3p, hsa-miR-6757-3p, hsa-miR-6889-3p, hsa-miR-6752-3p, hsa-miR-191-3p, hsa-miR-6795-3p, hsa-miR-149-5p, hsa-miR-2116-3p, hsa-miR-7974, hsa-miR-23c, hsa-miR-4310, hsa-miR-98-3p, hsa-miR-3190-5p, and/or hsa-miR-4312; and/or a binding partner for at least one of said miRNAs.

24. The microarray, solid support, or collection of solid supports of claim 22 comprising a nucleic acid amplification primer, a pair of nucleic acid amplification primers, and/or an oligonucleotide probe corresponding to at least one of said miRNAs.

25. The microarray, solid support, or collection of solid supports of claim 22 or 23 comprising SEQ ID NOS. 1-100; and/or, hsa-miR-4485-5p, hsa-miR-551b-3p, hsa-miR-24-1-5p, hsa-miR-6819-3p, hsa-miR-1238-3p, hsa-miR-6737-3p, hsa-miR-1237-3p, hsa-miR-6757-3p, hsa-miR-6889-3p, hsa-miR-6752-3p, hsa-miR-191-3p, hsa-miR-6795-3p, hsa-miR-149-5p, hsa-miR-2116-3p, hsa-miR-7974, hsa-miR-23c, hsa-miR-4310, hsa-miR-98-3p, hsa-miR-3190-5p, and hsa-miR-4312; and/or a binding partner for at least one of said miRNAs.

26. The solid support or collection of solid supports of any one of claims 22-24 wherein said solid support is a bead or collection of beads, respectively.

27. A kit comprising a component, microarray, solid support, or collection of solid supports or any one of claims 20-25, optionally further including instructions for use.
